(19) United States
(12) Patent Application Publication
(10) Pub. No.: US 2026/0200969 A1
(43) Pub. Date: Jul. 16, 2026

(54) Methods for Synthesis of Protein Hetero-polymers

(71) Applicant: The Regents of the University of California, Oakland, CA (US)

(72) Inventors: Alanna Schepartz Shrader, Berkeley, CA (US); Leah Tang Roe, Berkeley, CA (US)

(73) Assignee: The Regents of the University of California, Oakland, CA (US)

(21) Appl. No.: 19/567,124

(22) Filed: Mar. 14, 2026

Related U.S. Application Data
(63) Continuation of application No. PCT/US24/48032, filed on Sep. 23, 2024.
(60) Provisional application No. 63/587,179, filed on Oct. 2, 2023.

(51) Int. Cl. C07K 1/107; C12N 9/00; C12P 21/00

(52) U.S. Cl. CPC C07K 1/1075; C12N 9/93; C12P 21/00; C12Y 601/01026

(57) ABSTRACT

A method for synthesis of protein hetero-polymers uses an intramolecular Backbone Extension Acyl Rearrangement (BEAR) reactions to orthogonally and site-specifically embed a β-, γ- or δ-amino acid residue into proteins.

### CROSS-REFERENCES TO RELATED APPLICATIONS

This application is a continuation of PCT/US24/48032; filed: Sep. 23, 2024, which claims priority to U.S. Provisional Application No. 63/587,179; filed: Oct. 2, 2023, the disclosures of which are hereby incorporated by reference in its entirety for all purposes.

### GOVERNMENT SUPPORT CLAUSE

This invention was made with government support under grant 2002182 awarded by the National Science Foundation. The government has certain rights in the invention.

### INTRODUCTION

There is widespread current interest in the cellular biosynthesis of proteins and polypeptides whose backbones differ from those in extant proteins¹. Even single-atom substitutions, such as the introduction of an ester²⁻⁸ or thioester⁹⁻¹¹ in place of an amide, can promote new chemistry¹², facilitate mechanistic studies², and generate materials with emergent properties¹³⁻¹⁵. The simplest α-peptide backbone modification, beyond a single-atom substitution¹⁶, is the addition of one or more methylene (CH₂) units to generate β-, γ- or δ-amino acid linkages (FIG. 1A). Such expanded backbone linkages are replete in natural products, such as taxol¹⁷, andrimid¹⁸, and guineamide A¹⁹, that possess profound biological activity. Oligomers containing one or more extended backbone linkages can function as protein-protein interaction inhibitors²⁰⁻²⁷, antimicrobial agents²⁸⁻³⁴, receptor agonists and antagonists³⁵⁻⁴⁹, catalysts^{50,51}, and other useful tools. Certain extended backbone containing oligomers assemble into structurally defined protein-like materials⁵²⁻⁵⁷ that fold cooperatively and with enhanced thermal stability^{53,54} Even a small number of judiciously placed extended backbone-linkages within a protein add value, such as improved protease resistance⁵⁸⁻⁶², enhanced cellular permeability^{61,63-65}, and altered recognition when presented by major histocompatibility complex (MHC) receptors^{66,67}.

Despite the gain in function provided by extended backbone linkages, there exist only a few examples 68-72 in which an extended backbone monomer has been incorporated into a protein in vivo. In vitro translation systems containing wild type ribosomes can introduce certain extended backbone monomers into polypeptides73,74, but this methodology is not easily scalable and relies on stoichiometric RNA acylation reagents that have not been shown to function in cells. Indeed, new evidence suggests that complex engineering of EF-Tu, tRNA, and the ribosome may be needed to efficiently biosynthesize proteins containing even a single β²- and β³-amino acid linkage in E. coli^{7,75-77}. Here we describe an alternative approach to the programmed cellular synthesis of extended backbone-containing proteins that requires engineering of neither EF-Tu, tRNA, nor the ribosome, and supports the introduction of multiple extended backbone monomers in a single protein.

### SUMMARY OF THE INVENTION

The invention provides methods for synthesis of protein hetero-polymers uses an intramolecular Backbone Extension Acyl Rearrangement (BEAR) reactions to orthogonally and site-specifically embed a β-, γ- or δ-amino acid residue into full length proteins in vivo.

In aspects and embodiments the invention provides:
1. A method of protein backbone editing, the method comprising:
a) providing a cell engineered to express an orthogonal aminoacyl-tRNA synthetase under conditions wherein the synthetase accepts an α-hydroxy acid or α-thiol acid monomer comprising a side chain bearing a masked nucleophile, and the monomer is incorporated into a protein translated in the cell,
b) post-translationally unmasking the nucleophile of the incorporated monomer, wherein the unmasked monomer engages in a thermodynamically favored intramolecular Backbone Extension Acyl Rearrangement (BEAR) reaction that edits the protein backbone.
2. The method of claim 1, wherein the protein comprises a non-α-backbone comprising a non-α-peptide linkage, including an additional R group on a sidechain (shown in FIG. 1A) or an alternative hetero-atom (e.g. S, P, O, N) in the linkage.
3. The method of claim 1, wherein the BEAR reaction is used to generate a protein containing one or two copies of a β-backbone, a γ-backbone, or a δ-backbone.
4. The method of claim 1, wherein the protein comprises a β2-backbone comprising a β2-peptide linkage.
5. The method of claim 1, wherein the protein comprises a γ-backbone comprising a γ-peptide linkage.
6. The method of claim 1, wherein the protein comprises a δ-backbone comprising a δ-peptide linkage.
7. The method of claim 1, wherein the protein comprises a plurality of peptides linkages selected from β2-, γ- and δ-peptide linkages.
8. The method of claim 1, wherein the aminoacyl-tRNA synthetase is an orthogonal synthetase which incorporates the BEAR monomer that is an alpha-hydroxy acid with a nucleophile on the side chain for a BEAR reaction.
9. The method of claim 1, wherein the aminoacyl-tRNA synthetase is a pyrrolysyl-tRNA synthetase (PylRS).
10. The method of claim 1, wherein the aminoacyl-tRNA synthetase is a pyrrolysyl-tRNA synthetase (PylRS) of an archaea or methanogenic bacteria, wild-type or engineered.
11. The method of claim 1, wherein the aminoacyl-tRNA synthetase is a pyrrolysyl-tRNA synthetase (PylRS) of Methanosarcina mazei (Mm), Methanosarcina barkeri (Mb) or Methanocarcina Alvus (Ma), wild-type or engineered.
12. The method of claim 1, wherein the aminoacyl-tRNA synthetase is selected from:
highly polyspecific pyrrolysyl-tRNA synthetase” (HpRS) (e.g. Hohl et al. 2019, Scientific Reports 9, 11971), chimeric orthogonal aminoacyl-tRNA synthetase/tRNA pair (e.g. Ding et al, 2020, Nat Comm 11, 3154), Mb PylRS variant DAPRS (e.g. Huguenin-Dezot, et al. 2019 Nature. 565(7737):112-117) and engineered aminoacyl-tRNA synthetases reviewed in Gong et al. 2023, J Bacteriol. 205, 2, also, Koch N G, et al. Engineering Pyrrolysyl-tRNA Synthetase for the Incorporation of Non-Canonical Amino Acids with Smaller Side Chains, International Journal of Molecular Sciences, 2021; 22(20):11194.
13. The method of claim 1, wherein the nucleophile is a carbon or nitrogen atom, including secondary and tertiary amine and carbon nucleophiles.
14. The method of claim 1, wherein the unmasking is effected with light, heat, pH, reduction, oxidation, click chemistry, and other forms of biologically compatible chemical reactions.
15. The method of claim 1, wherein the masking is effected with photomasking or chemical reductive masking.
16. The method of claim 1, wherein the cell is bacterial (e.g. E. coli), yeast (e.g. S. cerevisiae) or mammalian (e.g. human).
17. The method of claim 1, wherein the cell is E. coli, S. cerevisiae or human.
18. A method of protein backbone editing, the method comprising:
using a proximity-guided intramolecular rearrangement that edits the protein backbone post-translationally, wherein an orthogonal aminoacyl-tRNA synthetase enzyme accepts diverse monomers, including those whose side chains contain masked nucleophiles,
wherein a monomer whose side chains contains a masked nucleophile is introduced into the protein translated in vivo, followed by nucleophile unmasking, to provide a thermodynamically favored intramolecular Backbone Extension Acyl Rearrangement (BEAR) reaction that edits the protein backbone to install an extended backbone monomer.
19. The method of claim 18, wherein the protein comprises a non-α-backbone comprising a non-α-peptide linkage, including an additional R group on a sidechain or an alternative hetero-atom, selected from S, P, O and N, in the linkage.
20. The method of claim 18, wherein the BEAR reaction is used to generate a protein containing one or two copies of a β-backbone, a γ-backbone, or a δ-backbone.

The invention encompasses all combinations of the particular embodiments recited herein, as if each combination had been laboriously recited.

### BRIEF DESCRIPTION OF THE DRAWINGS

FIGS. 1A-C. Backbone Extension Acyl Rearrangement (BEAR) reactions create extended backbones in full-length proteins expressed in vivo. (A) Structures of α-, β²-, γ- and δ-amino acids as well as photo-masked (pm) analogs of diaminopropionic acid (pm-DAP, 1), isoS (pm-isoS, 2), isoC (pm-isoC, (±)-3), and chemically masked monomers N3-isoT 4 and N3-AHP 5. (B) A BEAR reaction results from the intramolecular cyclization of an unmasked nucleophile, in this case, a primary amine, on an N-terminal ester. Breakdown of the resultant tetrahedral intermediate generates an extended backbone linkage embedded within the protein chain. PDB ID 2β3P. (C) BEAR reactions are analogous to the second step of the well-known native chemical ligation (NCL) reaction9,81.

FIGS. 2A-B. Computational analysis suggests that BEAR reactions are thermodynamically favorable. (A) We compared the energetics of intramolecular cyclization reactions that represent the second step of the canonical NCL reaction (top) and the single step BEAR reaction with 2, 3, or 4 carbons. Both were evaluated using simplified N-Me amide derivatives. The species [Intermediate] represents the cyclic intermediate formed during each acyl-transfer step. (B) Relative thermodynamics (ΔG°) of the NCL and BEAR reactions established using DFT (ωβ97M-V/6-311++G (3d,f) (3d,p), with a CPCM solvent model). Each reaction can proceed through two diastereomeric intermediates; only the lowest energy species is listed.

FIGS. 3A-I. BEAR reactions generate β²-peptide linkages in NanoLuc in vivo (A) Scheme illustrating the utility of pm-isoS 2 or pm-isoC (±)-3 for intra-protein BEAR reactions. Cellular incorporation of pm-isoS 2 or pm-isoC (±)-3 generates a protein containing an internal ester or thioester but retains a natural peptide-like backbone. Photo-unmasking generates an intermediate capable of a BEAR reaction to generate a β²-peptide linkage. PDB ID 7SNS. (B) Luminescence at the 18 h time point as a function of the concentration of pm-DAP 1, pm-isoS 2, or pm-isoC (±)-3 as well as Bock, K, and in the absence of any added substrate. (C) Coomassie-stained gel showing SDS-PAGE of NanoLuc affinity-purified from growths of DH10B E. coli expressing DAPRS and NanoLuc with a TAG codon inserted between positions G159 and V160 and supplemented with 1 mM pm-DAP 1 or pm-isoS 2. (D) Deconvoluted mass following LC-MS analysis of the affinity-purified NanoLuc G159-pm-isoS-V160 construct. The mass expected for NanoLuc containing pm-isoS 2 at a single position is 20,301 Da; the observed mass is 20,303. (E) Seven samples (Samples A-G) of NanoLuc were isolated from DH10B E. coli growths containing DAPRS and the monomers shown either before or after photo-unmasking. All were analyzed by high-resolution tryptic mapping. (F) Peptide mapping reveals partial photo-unmasking from ambient light prior to 370 nm irradiation, and full photo-unmasking upon direct exposure to 370 nm light. (G) Trace illustrating the coelution of the Sample D NanoLuc tryptic fragment comprising residues 155-165 (including the expected site of incorporation of pm-isoS 2) alongside authentic standard 10. The small later-eluting peak contains α-Ser. (H) The MS/MS profile of Sample D after tryptic digestion is identical to that of (I) authentic standard 10, providing conclusive evidence for a successful BEAR reaction. For peptide mapping, V160 in the native protein will be labeled as V161 and X will be in position 160.

FIGS. 4A-H. Efficient incorporation of N3-isoT 4 into SUMO-GFP in vivo. (A) Scheme illustrating the utility of N3-isoT 4 for intra-protein BEAR reactions. Cellular incorporation of N3-isoT 4 generates a protein containing an internal α-ester in the backbone. Chemical unmasking generates an intermediate capable of a BEAR reaction to generate a γ-peptide linkage. PDB ID 2B3P. (B) Plot showing emission at 528 nm (F528, emission maximum of GFP) at the 24 h time-point as a function of the concentration of N3-isoT 4 (0-15 mM) in both C321. ΔA.exp (DE3) and BL21(DE3) E. coli. (C) Coomassie-stained gel showing SDS-PAGE of SUMO-GFP affinity-purified from growths of C321. ΔA.exp (DE3) E. coli expressing PyIRS N311M: C313W and SUMO-GFP with a TAG codon at position 103 and supplemented with 10 mM N3-isoT 4. (D) Deconvoluted mass following LC-MS analysis of affinity-purified SUMO-GFP-103TAGN3-isoT product. The mass expected for SUMO-GFP containing N3-isoT 4 at a single position is 38994 Da; the observed mass is 38995. (E) Peptide mapping reveals quantitative unmasking upon exposure to 50 mM PTA for 2 h at 0 C and 2 h at 42 C at pH 8.2. (F) Trace illustrating the coelution of the SUMO-GFP tryptic fragment from the red boxed sample in D at the expected site of incorporation of isoT 4 alongside authentic standard A (QIGGM-isoT-KGEE). (G) The MS/MS profile of SUMO-GFP103isoT tryptic fragment is identical to that of (H) authentic standard A, providing conclusive evidence for a successful BEAR reaction.

FIGS. 5A-H. Efficient incorporation of N3-AHP 5 into SUMO-GFP in vivo. (A) Scheme illustrating the utility of Y for the biosynthesis of proteins containing one or more δ-peptide linkages. PDB ID 2B3P. (B) Plot showing emission at 528 nm (F₅₂₈, emission maximum of GFP) at the 24 h time-point as a function of the concentration of N3-AHP 5 (0-15 mM) in C321. ΔA.exp (DE3) and BL21 (DE3) E. coli. (C) Coomassie-stained SDS-PAGE gel of affinity-purified SUMO-GFP from growths of BL21 (DE3) E. coli expressing PyIRS N311M: C313W and SUMO-GFP with a TAG codon at position 103 and supplemented with 5 mM N3-AHP 5. (D) Deconvoluted mass spectrum of affinity-purified SUMO-GFP-103TAG expressed in the presence of N3-AHP 5. The mass expected for SUMO-GFP containing N3-AHP 5 at a single position is 39010 Da; the observed mass is 39010. (E) Peptide mapping reveals conversion to AHP at the 103 position of SUMO-GFP upon exposure to 50 mM PTA for 48 h at 50 C at pH 8.2. (F) Plot showing emission at 528 nm (F528, emission maximum of GFP) at the 24 h time-point as a function of the concentration of N3-AHP 5 (0-15 mM) in both C321. ΔA.exp (DE3) and BL21 (DE3) E. coli. In 3 double incorporation constructs: SUMO-GFP-103TAG-314TAG, SUMO-GFP-103TAG-295TAG, and SUMO-GFP-103TAG-289TAG (G) Coomassie-stained gel showing SDS-PAGE of SUMO-GFP affinity-purified from growths of BL21 (DE3) E. coli expressing PyIRS N311M: C313W and SUMO-GFP with a TAG codon at position 103 and 295 and supplemented with 5 mM N3-AHP 5. (E) Deconvoluted mass following LC-MS analysis of affinity-purified SUMO-GFP-103TAGN3-AHP-295TAGY product. The mass expected for SUMO-GFP containing N3-AHP 5 at a single position is 39150 Da; the observed mass is 39150.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

We disclose a proximity-guided intramolecular rearrangement that edits the protein backbone post-translationally. The method relies on the ability of orthogonal aminoacyl-tRNA synthetase enzymes to accept diverse monomers, including those whose side chains contain masked nucleophiles. Introduction of such a monomer into a protein translated in vivo, followed by nucleophile unmasking, sets up a thermodynamically favored intramolecular Backbone Extension Acyl Rearrangement (BEAR) reaction that edits the protein backbone to install an extended backbone monomer. In the examples described here, BEAR reactions are used to generate proteins containing one or two copies of a β-backbone, a γ-backbone, or a δ-backbone.

### EXAMPLES

### Backbone Extension Acyl Rearrangements Enables Protein Backbone Editing

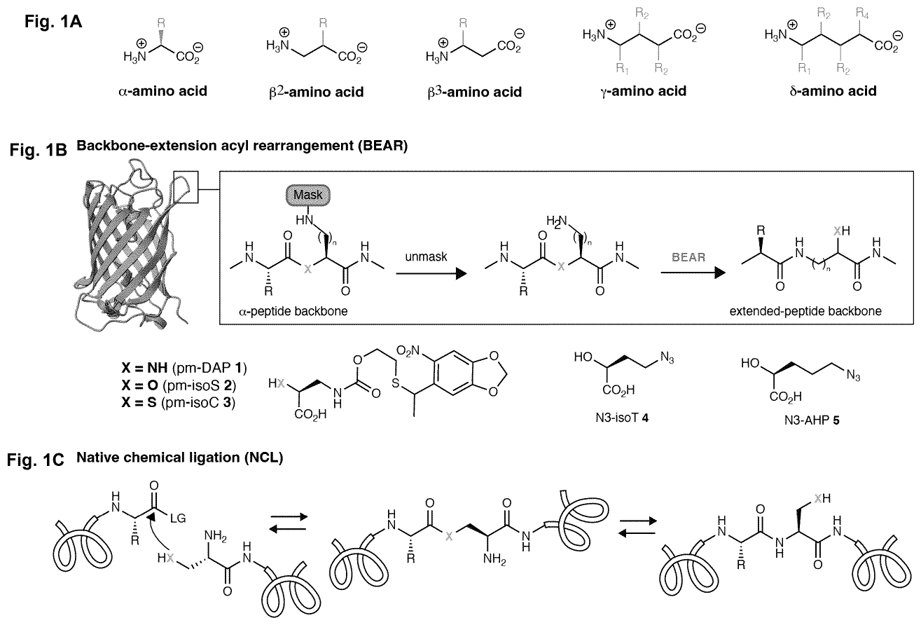

Rather than relying on direct reaction of an extended backbone monomer within the ribosomal peptidyl-transferase center (PTC) 68, the strategy reported here relies on a proximity-guided intramolecular Backbone Extension Acyl Rearrangement (BEAR) that effectively edits the protein backbone post-translationally to orthogonally and site-specifically install an extended backbone residue into full-length proteins in vivo. BEAR reactions rely on the fact that α-hydroxy acids are excellent substrates4,5,7,8 for pyrrolysyl-tRNA synthetase (PylRS), the aminoacyl-tRNA synthetase that naturally incorporates pyrrolysine into proteins in certain archaea and methanogenic bacteria, and is also orthogonal in E. coli. Recent work has shown that α-thiol acids⁷ are also substrates for certain PylRS variants. Furthermore, PylRS variants accept α-amino acids with a variety of side chains⁷⁸, including those carrying protected nucleophiles^{79,80} We envisioned that if an α-hydroxy or α-thiol substrate for such a PylRS variant carried a side chain bearing a masked amine nucleophile, that nucleophile could be unmasked post-translationally to promote a BEAR reaction (FIG. 1B), analogous to the second step of the native chemical ligation (NCL) reaction^{9,81} (FIG. 1C). The result of a BEAR reaction would be an internal extended backbone linkage.

Related acyl rearrangements within short peptides are known. It was reported in 2004 that isocysteine (isoC) supports an NCL-like reaction to install a β²-linkage into a chemically synthesized short peptide in vitro82. More recently, it was shown that backbone rearrangements involving isoserine (isoS), isothreonine (isoT), and 5-amino-2-hydroxypentanoic acid (AHP) can occur in short peptides prepared during small-scale in vitro translation reactions 12. Inspired by these findings, we report that proteins containing either a photo-masked isoS derivative (pm-isoS 2), or a chemically masked isoT (N3-isoT 4) and AHP derivative (N3-AHP 5), can be unmasked post-translationally, either in cells or after protein purification. Unmasking reveals a β-, γ-, or δ-amine nucleophile and triggers a spontaneous, intramolecular BEAR reaction that directly installs a β²-, γ- or δ-amino acid linkage. The installation of γ- and δ-amino acid backbones proceed with exceptional fidelity and yield, in the latter case supporting the biosynthesis of protein containing two such linkages in the same protein. This work represents a new, generalizable strategy to install extended backbones into proteins, and relies only on established activities of known orthogonal enzymes and the wild type E. coli ribosome. The hetero-oligomers so-generated expand the diversity of protein-like polypeptides that may be synthesized ribosomally.

Computational Studies Confirm that BEAR Reactions are Thermodynamically Favorable

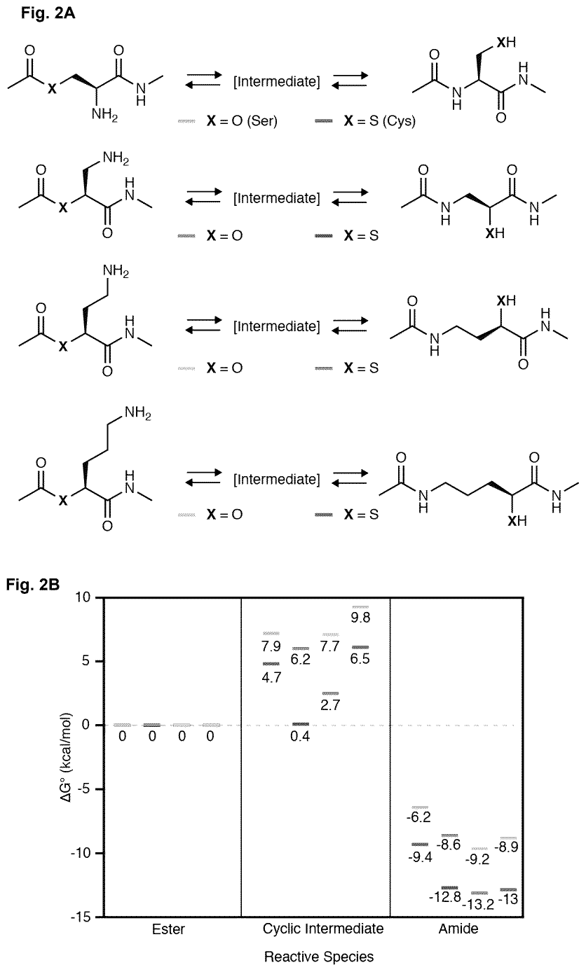

We began by performing computational studies to confirm that the proposed intramolecular rearrangements were thermodynamically plausible. A previous study made use of Density Functional Theory (DFT) to examine the energetics and mechanistic pathways of a canonical cysteine-based NCL reaction, but this study did not include any of the analogs considered herein83. For our analysis, we used DFT calculations to evaluate the relative energetics of acyl rearrangement reactions involving the natural α-amino acids Cys and Ser, their isomers isoC and isoS, as well as C3-OH BEAR, C3-SH-BEAR, C4-OH-BEAR and C4-SH BEAR nucleophiles in the context of a simplified substrate (FIG. 2A). For each proposed species in the reaction, molecular mechanics methods with an OPLS4 force field were first used to generate and evaluate 10,000 starting conformers for each species. The geometries of all conformers with energies within 5 kcal/mol of the global minimum were then re-optimized using DFT methods (r²SCAN-3c/mTZVPP with a CPCM water model). Following this, all conformers within 2 kcal/mol of the new global minimum were subjected to single point calculations to determine the vibrational frequencies at the r²SCAN-3c/CPCM level. This analysis allowed estimation of the zero-point energy and internal entropy parameters. Finally, single point electronic energies were determined with greater accuracy using ωB97M-V/def2-TZVPPD. These final calculations were performed with a SMD solvent model to represent aqueous solvation.

These calculations revealed that all of the proposed rearrangement reactions are thermodynamically plausible, as expected, but also identified unexpected differences as a function of intermediate ring size as well as heteroatom location and identity. We note that the BEAR reactions that generate β-, γ-, and δ-peptide linkages are more energetically favorable (by 2-3 kcal/mol) than the canonical NCL rearrangements of Cys and Ser in terms of both enthalpy and free energy (FIG. 2B). This improvement is likely due to increased hydrogen bonding in the BEAR products and relief of steric congestion. BEAR reactions that generate β-, γ-, and δ-peptide linkages are also more disfavored entropically than the canonical NCL rearrangements of Cys and Ser. This difference is especially true for the intermediate species. Of particular note, this leads to a high free energy intermediate for the δ-amino acid forming series. Nevertheless, due to their high overall energetic favorability, we reasoned that all of the proposed BEAR reactions could take place spontaneously in the context of an intact protein or translation product.

The validity of DFT calculations were verified by evaluating the intramolecular cyclization of O-to-N and S-to-N BEAR reactions to generate β²-backbone products in model systems under conditions that mimic those found in cells. In the model systems, pm-isoS 2 or pm-isoC (±)-3 were acylated with N-acetylglycine to generate ester 4 or thioester (±)-5, respectively. We also synthesized the authentic products 6 and (±)-7 that would result from the BEAR reaction of ester 4 or thioester (±)-5, respectively. The course of the BEAR reaction was monitored by LC-MS and product identity was confirmed by 1D- and 2D-NMR. A different reaction course was observed when thioester (±)-5 was incubated under identical conditions. Evaluation of the reaction components using LC-HRMS before UV irradiation revealed rapid hydrolysis of the thioester (t_{1/2<1} hour) to form thiol (±)-8 and generation of the analogous disulfide 9.

### BEAR Reactions Using Photo-Masked Amines Generate β²-Peptide Linkages In Vivo

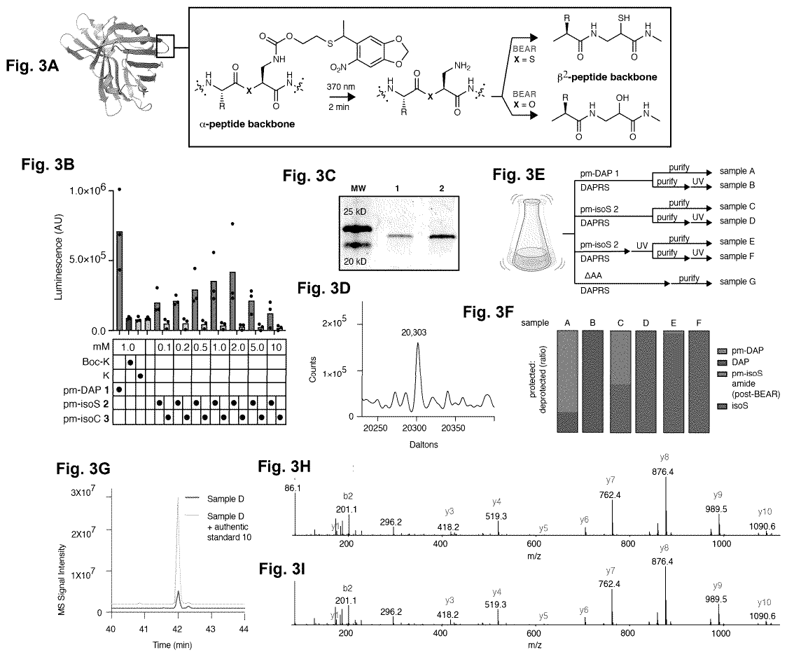

Given the computational data and experimental feasibility of model BEAR reactions to establish β²-linkages, we next asked whether these reactions could occur within full length proteins expressed in cells. We chose initially to photo-mask the β²-amine nucleophile, as photo-unmasking is compatible with cells and photo-masked diaminopropionic acid (pm-DAP) 1 (FIG. 1A) is a known substrate for the PylRS variant DAPRS79,84. We hypothesized that the analogous photo-masked isoserine (pm-isoS, 2) and photo-masked isocysteine (pm-isoC, (±)-3) would also act as DAPRS substrates and facilitate BEAR reactions in cells or after protein purification. We synthesized photo-masked isoserine (pm-isoS, 2), photo-masked isocysteine (pm-isoC, (±)-3), and the previously reported α-amino acid, pm-DAP (1). We first determined whether pm-isoS 2 or pm-isoC (±)-3 could be introduced at a single position within superfolder green fluorescent protein (sfGFP). Once incorporated, irradiation would unmask the nucleophilic amine that would promote the same intramolecular BEAR reaction observed in the model system to generate a β²-peptide linkage in situ (FIG. 3A). We focused initially on position N150 of sfGFP, which faces outward from the folded β-barrel and has previously been shown to incorporate pm-DAP 179.

To begin, we transformed DH10B E. coli cells with two plasmids, one encoding DAPRS/tRNA^{Pyl}_{CUA} (pBK-DAPRS) and a second encoding the N150TAG variant of sfGFP (p15A-sfGFP-N150TAG). Cells were grown in the presence of increasing concentrations (0.1-10 mM) of pm-isoS 2 or pm-isoC (±)-3. Control cultures supplemented with 1 mM pm-DAP 1 (positive control) or 1 mM Lys or Boc-Lys (negative controls) were run alongside, as well as a final control culture lacking any substrate. All growths were performed initially at 200 μL scale at 37° C. in a 96-well plate format. The change in OD₆₀₀ and fluorescence at 528 nm (F528) were evaluated over 24 h after induction with 0.2% arabinose when the OD₆₀₀ of the growths reached 0.6.

Examination of the fluorescence at 528 nm of each growth at the 24 h time-point indicated robust incorporation of pm-DAP 1, as expected, along with modest incorporation of pm-isoS 2. There was no evidence for incorporation of pm-isoC (±)-3. In the case of pm-isoS 2, emission due to GFP increased in a concentration-dependent manner with the highest GFP fluorescence observed at pm-isoS 2 concentrations between 0.2 mM and 2 mM. However, the overall level of sfGFP fluorescence was, at best, only 1.4-fold over background. We concluded that a more sensitive reporter protein was needed to optimize the incorporation of pm-isoS 2 prior to evaluating how to best implement a BEAR reaction and establish the desired β²-peptide linkage. The lack of apparent activity of pm-isoC (±)-3 is consistent with the previously reported lower activity of α-thiol acids as substrates for PylRS variants in vitro7. A purified sample of sfGFP containing pm-DAP 1 was used to optimize conditions needed for photo-unmasking.

We selected Nanoluciferase (NanoLuc) 85 as a more sensitive reporter, as its readout (i.e., light) is generated catalytically. We prepared a series of reporter plasmids with NanoLuc in place of sfGFP and TAG codons at six different positions. Of the six plasmids, two contained either a mutated or inserted TAG codon near the NanoLuc N-terminus (G2TAG and G2-TAG-V386), two contained inserted TAG codons within a loop (G159-TAG-V160⁸⁷ and G103-TAG-V104), and two placed mutated TAG codons within a β-sheet (T130-TAG and G131-TAG). To validate the plasmids, BL21 (DE3) E. coli were co-transformed separately with each NanoLuc reporter plasmid and pEVOL-PyIRS⁷, which encodes the Methanosarcina alvus PylRS/tRNA^{Pyl}_{CUA} pair. In each case, the cells were grown for 18 h in the presence of either 1 mM Bock or α-OH-Bock⁴ (positive controls), or Lys or no substrate (negative controls). After 18 h, luminescence was determined using the Nano-Glo® Luciferase Assay System (Promega). These experiments clearly identified the reporter encoding G159-TAG-V160 NanoLuc as possessing the largest dynamic range; the signal generated for growths containing α-OH-BocK was more than three orders of magnitude higher than growths to which no substrate had been added. All other constructs show a dynamic range of two orders of magnitude or less.

With an optimized detection system in hand, we next probed for in vivo incorporation of pm-isoS 2 or pm-isoC (±)-3. We transformed DH10B E. coli with pET15a-NanoLuc (G159-TAG-V160) as well as pBK-DAPRS encoding DAPRS/tRNA^{Pyl}_{CUA}, and grew the cells in the presence of 0.1 to 10 mM of pm-isoS 2 or pm-isoC (±)-3. Control cultures contained either 1 mM pm-DAP 1 (positive control) or 1 mM Lys, Bock, or no substrate (negative controls). The luminescence intensity of each growth was measured after 18 h as described above (FIG. 3B). Even with this more sensitive reporter, the photo-masked pm-isoC 3 showed no evidence of incorporation, with no signal above background at any substrate concentration. However, addition of pm-isoS 2 led to robust concentration-dependent NanoLuc expression, with up to 5.2-fold enhanced signal above background at a concentration of 2 mM 2 (FIG. 3B). LC-MS analysis of intact NanoLuc purified from a preparative-scale growth (FIG. 3C) showed a mass corresponding to the introduction of pm-isoS 2 at a single position (FIG. 3D).

We then subjected a purified sample of NanoLuc containing pm-isoS 2 to optimized unmasking conditions established using sfGFP containing pm-DAP 1 Although intact protein LC-MS analysis provided evidence that pm-isoS 2 had been introduced into NanoLuc and unmasked, this technique alone could not confirm that a BEAR reaction had occurred, as the BEAR reaction is fundamentally an isomerization. To confirm that the BEAR reaction occurred, purified NanoLuc samples from growths supplemented with pm-DAP 1 or pm-isoS 2 were unmasked and evaluated by high-resolution tryptic peptide mapping (FIG. 3E) alongside a synthetic authentic tryptic standard 10 containing the anticipated β²-substituted product of the BEAR reaction. Samples A and B (positive controls) were purified from growths supplemented with pm-DAP 1 (Sample A) and subsequently irradiated at 370 nm (Sample B). Samples C and D were purified from growths supplemented with pm-isoS 2 (Sample C) and subsequently irradiated (Sample D). Similarly, Sample E was isolated from a growth supplemented with pm-isoS 2, but in this case the cells were irradiated with 370 nm light prior to cell lysis and NanoLuc purification. Sample F followed the same procedure as sample E with an additional 370 nm irradiation step after purification. Sample G contained material from a NanoLuc expression to which no substrate was added as a negative control.

All samples were denatured and digested with trypsin prior to LC-MS/MS. Trypsin cleaves NanoLuc to generate a fragment containing residues V155 through R165 with an additional residue between G159 and V160. As anticipated, when isolated from Samples A and B, this tryptic fragment contained primarily either pm-DAP 1 (Sample A) or its unmasked product DAP (Sample B). Comparison of the compositions of Samples A and B demonstrate essentially quantitative unmasking (FIG. 3F) with approximately 30% Gln in place of pm-DAP 1 due to misincorporation.

NanoLuc isolated from growths supplemented with pm-isoS 2 generated analogous tryptic fragments. Tryptic mapping of Sample C revealed that 2.09% of the isolated NanoLuc contained pm-isoS 2 between G159 and V160, while tryptic mapping of Sample D revealed the isolated NanoLuc contained 0.0% pm-isoS 2 and 9.5% of a product whose mass corresponded to isoS-either the deprotected BEAR precursor or the anticipated BEAR product containing a β²-peptide linkage.

To determine if Sample D contained the anticipated BEAR product, we synthesized peptide 10, which contains the appropriate β²-residue between G159 and V160. When co-injected on LC-MS/MS, peptide 10 co-eluted with the NanoLuc tryptic product isolated from Sample D whose mass corresponded to isoS (FIG. 3G), and the two materials generated identical MS/MS spectra (FIGS. 3H and 3I). Notably, the tryptic product from Sample D whose mass corresponded to isoS does not co-elute with a synthetic peptide containing Ser between G159 and V160. We hypothesize that the lower level of pm-isoS 2 in Sample C is due to ester hydrolysis during sample preparation or LC-MS/MS analysis; photo-unmasking of Sample D provides protection against hydrolysis by creating a stable amide bond. Together, these experiments provide conclusive evidence that a protein containing pm-isoS 2 can be unmasked quantitatively and subsequently undergo a spontaneous intramolecular BEAR reaction to install a β²-peptide in the NanoLuc backbone.

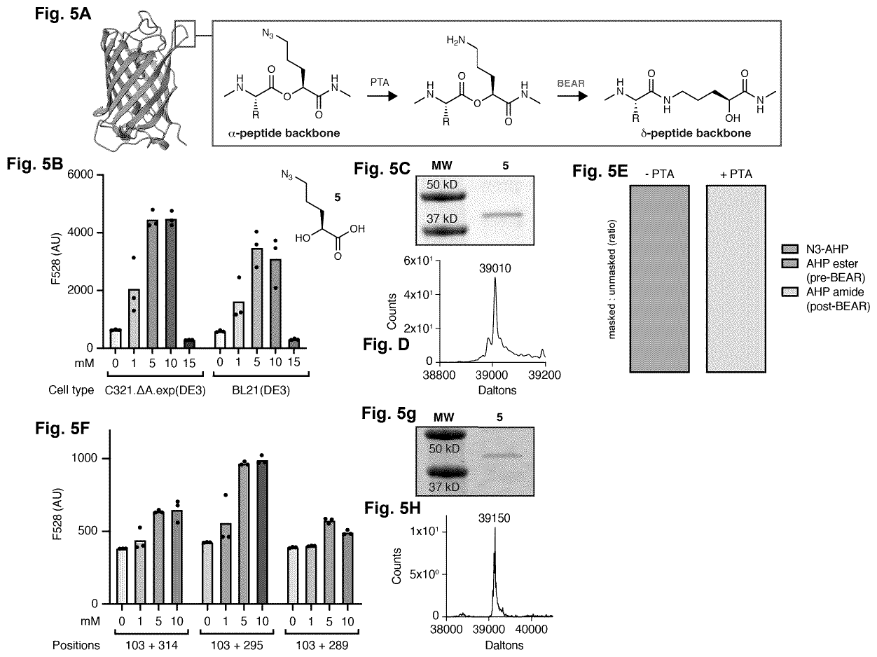

Analysis of the remaining samples provides insight into an optimal BEAR workflow for establishing β²-linkages within intact proteins. Tryptic samples derived from Sample E, where cells were irradiated prior to NanoLuc purification, contained 0.15% pm-isoS 2 and 5.79% of the BEAR product, indicating that higher levels of the rearranged β²-amino acid BEAR product are achieved with post-purification unmasking. Consistent with this notion, Sample F, which was irradiated before and after NanoLuc purification, contained 0.0% of pm-isoS 2 and 9.35% of the β²-amino acid BEAR product (FIG. 5C). We note that all NanoLuc samples expressed from growths containing pm-isoS 2 contained significant levels of Ala (~33%) and Gln (~22%) in place of pm-isoS 2. No evidence of pm-DAP 1, DAP, pm-isoS 2, or isoS was found in negative control Sample G.

### BEAR Reactions Using Reductively Masked Amines Generate γ-Linkages in High Yield

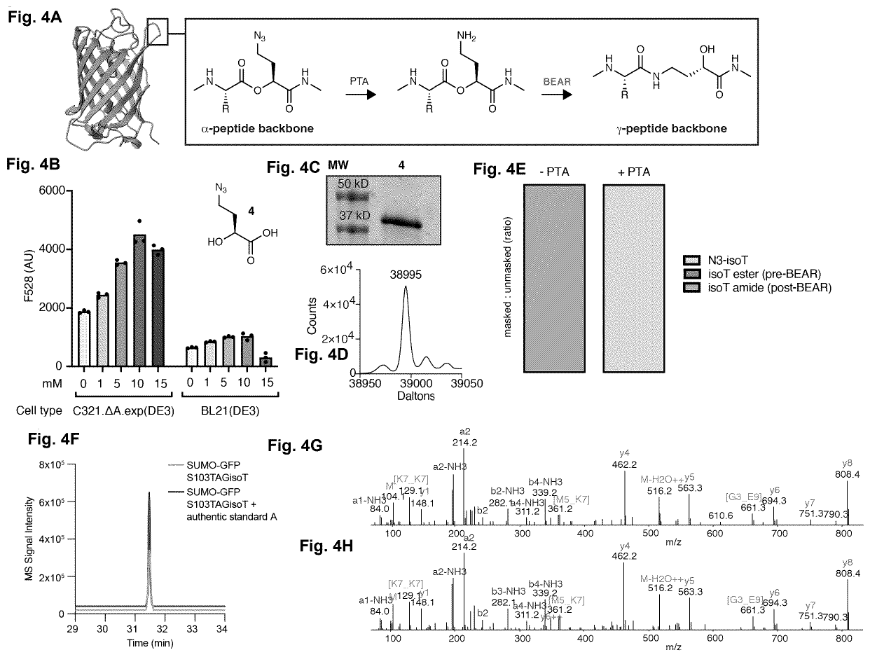

Proteins containing γ-amino acid linkages have never been produced in cells or in vitro in isolable quantities. Indeed, tRNAs acylated with γ-amino acids are prone to degradation via intramolecular cyclization reactions88, complicating their direct incorporation by wild type or remodeled ribosomes. As DFT calculations suggested that BEAR reactions to generate-peptide linkages are energetically favorable, we examined the incorporation of azide substrate N3-isoT 4, which we envisioned could be unmasked using a reducing agent (FIG. 4A). Previous work had shown that the α-amino versions of N3-isoT 4 is a substrate for variants of M. barkeri PylRS containing substitutions at two active site residues: 311 and 313 (PylRS N311M: C313W) 80. We transformed C321. ΔA.exp (DE3) and BL21 (DE3) E. coli with plasmids encoding SUMO-GFP-103TAG and PylRS N311M: C313W and grew the cells in the presence of increasing concentrations of N3-isoT 4, from 0 to 15 mM. Although only modest levels of GFP fluorescence were measured in growths of BL21 (DE3) cells at the 24 h time-point, growths of C321. ΔA.exp (DE3) cells showed substantial levels of GFP fluorescence over background when supplemented with 5-15 mM N3-isoT 4 (FIG. 4B). A preparative-scale growth of C321. ΔA.exp (DE3) E. coli expressing PyIRS N311M: C313W and SUMO-GFP-103TAG and supplemented with 10 mM N3-isoT 4 generated 10.2 mg/L of a protein whose mass corresponds to the introduction of N3-isoT 4 at a single position (FIGS. 4C and D).

Next we sought to establish conditions to reductively unmask SUMO-GFP containing N₃-isoT 4 and promote its intramolecular isomerization into a γ-peptide linkage, isoT. Peptide mapping was used to evaluate all reducing conditions. Two samples of SUMO-GFP-103N3-isoT isolated from C321. ΔA.exp (DE3) E. coli expressing PyIRS N311M: C313W and supplemented with 10 mM N3-isoT 4 with or without 50 mM of the reducing agent tris(2-carboxyethyl)phosphine (TCEP) and incubated for 16 h at RT in a pH 6.8 buffer. In the absence of TECP, the product contained more than 91% N₃-isoT 4 at position 103. After treatment with TCEP, the SUMO-GFP product contained virtually undetectable amounts of N3-isoT 4 at position 103. Instead, the sample was composed of a 1:2 mixture of two products: one whose mass corresponded to reduction of the azide to an amine, and another whose mass corresponded to conversion of the azide to an alcohol, a known side-product of phosphine-mediated azide reductions12.

In effort to minimize production of the alcohol side-product, we evaluated the amine to ester SUMO-GFP product ratio after reduction with both tris(3-hydroxypropyl)phosphine (THPP) and 1,3,5-Triaza-7-phosphaadamantane (PTA), as well as at various pH, times, and temperatures. Samples of SUMO-GFP containing a single residue of N3-isoT 4 were treated with 50 mM TCEP, THPP, or PTA in sodium phosphate buffer pH 8.5, tris buffer 9 or CAPS buffer 10 and at RT for 4 h, 42° C. for 4 h, or 0° C. for 2 h followed by incubation at 42° C. for 2 h. Samples reduced with 50 mM TCEP at pH 8.5 and 9 contained a 1:1 mixture of amine to alcohol. At pH 10 in 50 mM TCEP, virtually all of the alcohol side product was eliminated. Samples reduced with 50 mM THPP followed a similar trend. All samples reduced with 50 mM PTA virtually eliminated the alcohol side product. The most biologically relevant condition was identified as 50 mM PTA at pH 8.5 with incubation a 0° C. for 2 h then 42° C. for 2 h. This study identified PTA as the superior reducing agent, virtually eliminating the alcohol side product across all conditions.

We note that at pH 8.5 or above, ester hydrolysis competes with the intramolecular BEAR reaction. At pH 8.5, 9, and 10 with no reducing agent, we observed that 38%, 40%, and 79% (respectively) of the protein was hydrolyzed. With the addition of reducing agents, hydrolysis was minimized presumably due to the BEAR reaction taking place, replacing the unstable easter with a stable amide bond. Across all conditions, THPP samples generally had the highest amount of hydrolysis relative to samples in the same pH and temperature condition (3% to 46%) and PTA had the lowest (1% to 26%). As pH and temperature increased, hydrolysis also increased across samples (1% to 40% and 1% to 46%, respectively).

In an effort to reduce hydrolysis while maintaining a quantitative BEAR reaction, we conducted a final pH test using the best condition from the prior optimization. Samples of SUMO-GFP containing a single residue of N3-isoT X were treated with 50 mM PTA at pH 6.8, 7.2, 7.9, 8.2 and 8.5 at 0° C. for 2 h then 42° C. for 2 h. At pH6.8, we observe <1% hydrolysis but low BEAR conversion (50.6% ester and 34.3% amide). At pH 7.9 and 8.2, we observe high BEAR conversion (0.1% ester and 89.51% amide, 0.05% ester and 91% amide, respectively and <1% hydrolysis. The optimal pH was pH 8.2, which showed a quantitative BEAR reaction with minimal hydrolysis <1% hydrolysis (FIG. 4E). Co-injection experiments on an LC-MS/MS with an authentic standard of GlnC digest product containing residues 97-106

co-eluted with the SUMO-GFP GluC digest product (FIG. 4F), and the two materials generated identical MS/MS spectra (FIGS. 4G and H) confirming that reduction to the amine was accompanied by quantitative conversion into the γ-peptide product. Notably, the GluC product whose mass corresponded to isoS does not co-elute with a synthetic peptide containing Thr at position 103.

### BEAR Reactions Using Reductively Masked Amines Generate Multiple δ-Peptide Linkages in a Single Protein

We next sought to introduce N3-AHP 5, which upon reductive demasking and intramolecular cyclization would generate a single δ-peptide linkage, AHP, within a protein expressed in cells (FIG. 5A). Notably, cyclization in this case proceeds through a 7-membered ring intermediate, whose formation is less favorable than the 5- and 6-membered ring intermediates formed during BEAR reactions to form β- and γ-peptide linkages. Using the same transformed E. coli with plasmids encoding SUMO-GFP-103TAG and PyIRS N311M: C313W, we grew cells at N3-AHP N3-AHP 5 concentrations of 0-15 mM and monitored cell density and GFP fluorescence as a function of time. N3-AHP 5 is an exceptional substrate for PyIRS N311M:C313W in both C321. DA.exp(DE3) and BL21(DE3) cells (FIG. 5B). Negative control cultures contained no substrate.

A preparative growth of BL21 (DE3) E. coli expressing PyIRS N311M:C313W and SUMO-GFP with a TAG codon at position 103 and supplemented with 5 mM N3-AHP 5 generated 48.6 mg/L purified SUMO-GFP whose mass corresponded to the introduction of N3-AHP 5 at a single position (FIG. 5C, D). Next we sought to establish conditions to reductively demask sfGFP containing N3-AHP 5 and promote its intramolecular isomerization into a δ-peptide linkage. Based on optimized conditions from the γ-BEAR reaction, we chose to use 50 mM PTA at 0 C for 2 h then 42 C for 2 h at pHs 6.8, 7.2, 7.9, 8.2, and 8.5. In the absence of PTA, the product contained more than 83% N3-AHP 5 at position 103. After treatment with PTA, we found that similar to the 6-membered ring reaction, the percent of the BEAR product increased as pH increased up to pH 8.2, and then dipped down again as hydrolysis began competing. However, at pH 8.2 only 5.3% of the population contained the correct BEAR δ-peptide linkage product, with 73.2% still at the ester stage. We hypothesized that the 7-membered ring intermediate may take longer to form and thus explored six more conditions with 50 mM PTA at pH 8.2 at 42 C or 50 C for 8 h, 24 h, or 48 h. As temperature and time were increased, more of the δ-BEAR product was created with the best condition being pH 8.2 at 50 C for 48 h with a fidelity of 28% with no un-rearranged product remaining (FIG. 5E). The amide product may also be determined based on pH trends. We find that hydrolysis becomes a major competing reaction when the BEAR reaction is slow, resulting in a seemingly lower fidelity.

Because of the high protein yield and fidelity of SUMO-GFP containing N3-AHP 5, we next sought to incorporate N3-AHP 5 in two spots in SUMO-GFP. Three new SUMO-GFP constructs were created with TAG sites at positions 103 and 314, 295, or 289. BL21 (DE3) E. coli expressing PyIRS N311M: C313W and each of the three new double-TAG SUMO-GFP constructs were grown at concentrations of 0-15 mM N3-AHP 5 (FIG. 5F). We find that the best construct for double incorporation of N3-AHP 5 is SUMO-GFP-103TAG-295TAG, yielding the highest signal above background. A preparative growth of BL21 (DE3) E. coli expressing PyIRS N311M: C313W and SUMO-GFP-103TAG-295TAG and supplemented with 5 mM N3-AHP 5 generated 35 mg/L a protein with a mass corresponding to the introduction of N3-AHP 5 at a two positions (FIG. 5G, H).

### Some Conclusions

Despite the availability of multiple orthogonal aminoacyl-tRNA synthetase enzymes for non-canonical α-amino acids90,91, since the initial report in 201668 there have been only two additional examples which an extended backbone monomer has been introduced into a protein in cells, in each case a modestly extended β-backbone monomer^{71,72}. Although certain PylRS enzymes process monomers with β²- and β³-backbones, there are three challenges at work. One challenge is the ribosome, whose ability to promote efficient bond-forming reactions in vivo to and from anything other than an α-amino acid or α-hydroxy acid is unknown⁷⁵. The second is EF-Tu, whose ability to deliver tRNAs acylated with extended-backbone monomers is severely challenged. The third challenge is the availability of ribosome substrates—acylated tRNAs. Although recent work has expanded the diversity of monomers accepted by certain archaeal aaRS enzymes^{4,7}, there is still not a single efficient and orthogonal enzyme that acylates tRNA with anything other than a β²-hydroxy or β³-amino acid, let alone a monomer that differs from an α-amino acid by more than a single CH₂ group.

Here we bypass these challenges by reframing the problem of cellular hetero-oligomer synthesis in the language of chemistry. Rather than relying on direct reactions of extended backbone monomers within the ribosome active site, the strategy reported here relies on post-translationally initiated, proximity-guided intramolecular rearrangements that effectively “edit” the protein backbone. In the examples reported here, the intramolecular rearrangement converts an α-backbone into a β²-backbone, a γ-backbone, or a δ-backbone, in the final example at two non-adjacent positions simultaneously. As far as we know, this report represents the first example in which a single extended backbone β²-, γ- or δ-amino acid linkage has been introduced in an orthogonal fashion into a protein in a cell, and the first example of a cellular protein containing two δ-peptide linkages. Although we demonstrate this concept using relatively unadorned side chain nucleophiles, the strategy we describe is readily practiced with alternative nucleophiles, side chain geometries, and unmasking strategies capable of installing more diverse backbones into proteins expressed in cells and with temporal control.

### REFERENCES

1. Kwon, D. How scientists are hacking the genetic code to give proteins new powers. Nature 618, 874-876 (2023).
2. England, P. M., Zhang, Y., Dougherty, D. A. & Lester, H. A. Backbone Mutations in Transmembrane Domains of a Ligand-Gated Ion Channel: Implications for the Mechanism of Gating. Cell 96, 89-98 (1999).
3. Guo, J., Wang, J., Anderson, J. C. & Schultz, P. G. Addition of an α-Hydroxy Acid to the Genetic Code of Bacteria. Angew. Chem. Int. Ed. 47, 722-725 (2008).
4. Kobayashi, T., Yanagisawa, T., Sakamoto, K. & Yokoyama, S. Recognition of Non-α-amino Substrates by Pyrrolysyl-tRNA Synthetase. J. Mol. Biol. 385, 1352-1360 (2009).
5. Li, Y.-M. et al. Ligation of Expressed Protein α-Hydrazides via Genetic Incorporation of an α-Hydroxy Acid. ACS Chem. Biol. 7, 1015-1022 (2012).
6. Bindman, N. A., Bobeica, S. C., Liu, W. R. & van der Donk, W. A. Facile Removal of Leader Peptides from Lanthipeptides by Incorporation of a Hydroxy Acid. J. Am. Chem. Soc. 137, 6975-6978 (2015).
7. Fricke, R. et al. Expanding the substrate scope of pyrrolysyl-transfer RNA synthetase enzymes to include non-α-amino acids in vitro and in vivo. Nat. Chem. 15, 960-971 (2023).
8. Spinck, M. et al. Genetically programmed cell-based synthesis of non-natural peptide and depsipeptide macrocycles. Nat. Chem. 15, 61-69 (2023).
9. Dawson, P. E., Muir, T. W., Clark-Lewis, I. & Kent, S. B. H. Synthesis of Proteins by Native Chemical Ligation. Science (1994).
10. Muir, T. W., Sondhi, D. & Cole, P. A. Expressed protein ligation: A general method for protein engineering. Proc. Natl. Acad. Sci. 95, 6705-6710 (1998).
11. Grain, B., Desmet, R., Snella, B., Melnyk, O. & Agouridas, V. Incorporation of a Highly Reactive Oxalyl Thioester-Based Interacting Handle into Proteins. Org. Lett. 25, 5117-5122 (2023).
12. Kuroda, T., Huang, Y., Nishio, S., Goto, Y. & Suga, H. Post-translational backbone-acyl shift yields natural product-like peptides bearing hydroxyhydrocarbon units. Nat. Chem. 14, 1413-1420 (2022).
13. Ghadiri, M. R., et al. Self-assembling organic nanotubes based on a cyclic peptide architecture. Nature 366, 324-327 (1993).
14. Kwon, S. et al. Self-Assembled Peptide Architecture with a Tooth Shape: Folding into Shape. J. Am. Chem. Soc. 133, 17618-17621 (2011).
15. Yashima, E. et al. Supramolecular Helical Systems: Helical Assemblies of Small Molecules, Foldamers, and Polymers with Chiral Amplification and Their Functions. Chem. Rev. 116, 13752-13990 (2016).
16. Boger, D. L. The Difference a Single Atom Can Make: Synthesis and Design at the Chemistry-Biology Interface. J. Org. Chem. 82, 11961-11980 (2017).
17. Wani, M. C., Taylor, H. L., Wall, M. E., Coggon, P. & McPhail, A. T. Plant antitumor agents. VI. Isolation and structure of taxol, a novel antileukemic and antitumor agent from Taxus brevifolia. J. Am. Chem. Soc. 93, 2325-2327 (1971).
18. Fredenhagen, A. et al. Andrimid, a new peptide antibiotic produced by an intracellular bacterial symbiont isolated from a brown planthopper. J. Am. Chem. Soc. 109, 4409-4411 (1987).
19. Tan, L. T., Sitachitta, N. & Gerwick, W. H. The Guineamides, Novel Cyclic Depsipeptides from a Papua New Guinea Collection of the Marine Cyanobacterium Lyngbya majuscula. J. Nat. Prod. 66, 764-771 (2003).
20. Kritzer, J. A., Lear, J. D., Hodsdon, M. E. & Schepartz, A. Helical β-Peptide Inhibitors of the p53-hDM2 Interaction. J. Am. Chem. Soc. 126, 9468-9469 (2004).
21. Sadowsky, J. D. et al. Chimeric (α/β+α)-Peptide Ligands for the BH3-Recognition Cleft of Bcl-xL: Critical Role of the Molecular Scaffold in Protein Surface Recognition. J. Am. Chem. Soc. 127, 11966-11968 (2005).
22. Stephens, O. M. et al. Inhibiting HIV Fusion with a β-Peptide Foldamer. J. Am. Chem. Soc. 127, 13126-13127 (2005).
23. Bautista, A. D. et al. Identification of a β3-peptide HIV fusion inhibitor with improved potency in live cells. Bioorg. Med. Chem. Lett. 19, 3736-3738 (2009).
24. Horne, W. S. et al. Structural and biological mimicry of protein surface recognition by α/β-peptide foldamers. Proc. Natl. Acad. Sci. 106, 14751-14756 (2009).
25. Imamura, Y. et al. Inhibition of γ-Secretase Activity by Helical β-Peptide Foldamers. J. Am. Chem. Soc. 131, 7353-7359 (2009).
26. Imamura, Y. et al. Effect of Helical Conformation and Side Chain Structure on γ-Secretase Inhibition by β-Peptide Foldamers: Insight into Substrate Recognition. J. Med. Chem. 56, 1443-1454 (2013).
27. Miura, T. et al. In vitro selection of macrocyclic peptide inhibitors containing cyclic γ2,4-amino acids targeting the SARS-COV-2 main protease. Nat. Chem. 15, 998-1005 (2023).
28. Hamuro, Y., Schneider, J. P. & DeGrado, W. F. De Novo Design of Antibacterial β-Peptides. J. Am. Chem. Soc. 121, 12200-12201 (1999).
29. Liu, D. & DeGrado, W. F. De Novo Design, Synthesis, and Characterization of Antimicrobial β-Peptides. J. Am. Chem. Soc. 123, 7553-7559 (2001).
30. Arvidsson, P. I. et al. On the Antimicrobial and Hemolytic Activities of Amphiphilic β-Peptides. ChemBioChem 2, 771-773 (2001).
31. Porter, E. A., Wang, X., Lee, H.-S., Weisblum, B. & Gellman, S. H. Non-haemolytic β-amino-acid oligomers. Nature 404, 565-565 (2000).
32. Raguse, T. L., Porter, E. A., Weisblum, B. & Gellman, S. H. Structure-Activity Studies of 14-Helical Antimicrobial β-Peptides: Probing the Relationship between Conformational Stability and Antimicrobial Potency. J. Am. Chem. Soc. 124, 12774-12785 (2002).
33. Porter, E. A., Weisblum, B. & Gellman, S. H. Use of Parallel Synthesis To Probe Structure-Activity Relationships among 12-Helical β-Peptides: Evidence of a Limit on Antimicrobial Activity. J. Am. Chem. Soc. 127, 11516-11529 (2005).
34. Schmitt, M. A., Weisblum, B. & Gellman, S. H. Unexpected Relationships between Structure and Function in α,β-Peptides: Antimicrobial Foldamers with Heterogeneous Backbones. J. Am. Chem. Soc. 126, 6848-6849 (2004).
35. Cheloha, R. W., Maeda, A., Dean, T., Gardella, T. J. & Gellman, S. H. Backbone modification of a polypeptide drug alters duration of action in vivo. Nat. Biotechnol. 32, 653-655 (2014).
36. Urman, S. et al. The Constrained Amino Acid β-Acc Confers Potency and Selectivity to Integrin Ligands. Angew. Chem. Int. Ed. 46, 3976-3978 (2007).
37. Hayashi, Y. et al. GPIIb/IIIa Integrin Antagonists with the New Conformational Restriction Unit, Trisubstituted β-Amino Acid Derivatives, and a Substituted Benzamidine Structure. J. Med. Chem. 41, 2345-2360 (1998).
38. Tolomelli, A. et al. Modulation of αvβ3- and α5β1-integrin-mediated adhesion by dehydro-β-amino acids containing peptidomimetics. Eur. J. Med. Chem. 66, 258-268 (2013).
39. Peggion, E. et al. Structure-Function Studies of Analogues of Parathyroid Hormone (PTH)-1-34 Containing β-Amino Acid Residues in Positions 11-13, Biochemistry 41, 8162-8175 (2002).
40. Denton, E. V. et al. A β-Peptide Agonist of the GLP-1 Receptor, a Class B GPCR. Org. Lett. 15, 5318-5321 (2013).
41. Keresztes, A. et al. New Endomorphin Analogues Containing Alicyclic β-Amino Acids: Influence on Bioactive Conformation and Pharmacological Profile. J. Med. Chem. 51, 4270-4279 (2008).
42. Wilczyńska, D., Kosson, P., Kwasiborska, M., Ejchart, A. & Olma, A. Synthesis and receptor binding of opioid peptide analogues containing β3-homo-amino acids. J. Pept. Sci. 15, 777-782 (2009).
43. Wang, Y. et al. A New Class of Highly Potent and Selective Endomorphin-1 Analogues Containing α-Methylene-β-aminopropanoic Acids (Map). J. Med. Chem. 55, 6224-6236 (2012).
44. Seebach, D. et al. New Open-Chain and Cyclic Tetrapeptides, Consisting of α-, β2-, and β3-Amino-Acid Residues, as Somatostatin Mimics—A Survey. Helv. Chim. Acta 91, 1736-1786 (2008).
45. Seebach, D. et al. Linear, Peptidase-Resistant β2/β3-Di- and α/β3-Tetrapeptide Derivatives with Nanomolar Affinities to a Human Somatostatin Receptor, Preliminary Communication. Helv. Chim. Acta 84, 3503-3510 (2001).
46. Gademann, K., Ernst, M., Hoyer, D. & Seebach, D. Synthesis and Biological Evaluation of a Cyclo-β-tetrapeptide as a Somatostatin Analogue. Angew. Chem. Int. Ed. 38, 1223-1226 (1999).
47. Gademann, K., Kimmerlin, T., Hoyer, D. & Seebach, D. Peptide Folding Induces High and Selective Affinity of a Linear and Small β-Peptide to the Human Somatostatin Receptor 4. J. Med. Chem. 44, 2460-2468 (2001).
48. Seebach, D. et al. On the Terminal Homologation of Physiologically Active Peptides as a Means of Increasing Stability in Human Serum-Neurotensin, Opiorphin, B27-KK10 Epitope, NPY. Chem. Biodivers. 8, 711-739 (2011).
49. Koglin, N. et al. Analogues of Neuropeptide Y Containing β-Aminocyclopropane Carboxylic Acids are the Shortest Linear Peptides That Are Selective for the Y1 Receptor. Angew. Chem. Int. Ed. 42, 202-205 (2003).
50. Wang, P. S. P., Nguyen, J. B. & Schepartz, A. Design and High-Resolution Structure of a β3-Peptide Bundle Catalyst. J. Am. Chem. Soc. 136, 6810-6813 (2014).
51. Girvin, Z. C., Andrews, M. K., Liu, X. & Gellman, S. H. Foldamer-templated catalysis of macrocycle formation. Science 366, 1528-1531 (2019).
52. Daniels, D. S., Petersson, E. J., Qiu, J. X. & Schepartz, A. High-Resolution Structure of a β-Peptide Bundle. J. Am. Chem. Soc. 129, 1532-1533 (2007).
53. Qiu, J. X., Petersson, E. J., Matthews, E. E. & Schepartz, A. Toward β-Amino Acid Proteins: A Cooperatively Folded β-Peptide Quaternary Structure. J. Am. Chem. Soc. 128, 11338-11339 (2006).
54. Goodman, J. L., Petersson, E. J., Daniels, D. S., Qiu, J. X. & Schepartz, A. Biophysical and Structural Characterization of a Robust Octameric β-Peptide Bundle. J. Am. Chem. Soc. 129, 14746-14751 (2007).
55. Petersson, E. J. & Schepartz, A. Toward β-Amino Acid Proteins: Design, Synthesis, and Characterization of a Fifteen Kilodalton β-Peptide Tetramer. J. Am. Chem. Soc. 130, 821-823 (2008).
56. Guo, L. et al. Helix Formation in Preorganized β/γ-Peptide Foldamers: Hydrogen-Bond Analogy to the α-Helix without α-Amino Acid Residues. J. Am. Chem. Soc. 132, 7868-7869 (2010).
57. Sharma, G. V. M. et al. Design and Synthesis of Peptides with Hybrid Helix-Turn-Helix (HTH) Motif and Their Conformational Study. J. Org. Chem. 79, 8614-8628 (2014).
58. Seebach, D. et al. β-Peptides: Synthesis by Arndt-Eistert homologation with concomitant peptide coupling. Structure determination by NMR and CD spectroscopy and by X-ray crystallography. Helical secondary structure of a β-hexapeptide in solution and its stability towards pepsin. Helv. Chim. Acta 79, 913-941 (1996).
59. Seebach, D. et al. Biological and Pharmacokinetic Studies with β-Peptides. CHIMIA 52, 734-734 (1998).
60. Hintermann, T. & Seebach, D. The Biological Stability of β-Peptides: No Interactions between α- and β-Peptidic Structures?CHIMIA 51, 244-244 (1997).
61. Adaligil, E., Song, A., Cunningham, C. N. & Fairbrother, W. J. Ribosomal Synthesis of Macrocyclic Peptides with Linear γ4- and β-Hydroxy-γ4-amino Acids. ACS Chem. Biol. 16, 1325-1331 (2021).
62. Pasco, M., Dolain, C. & Guichard, G. 5.05—Foldamers in Medicinal Chemistry. in Comprehensive Supramolecular Chemistry II (ed. Atwood, J. L.) 89-125 (Elsevier, Oxford, 2017). doi: 10.1016/β978-0-12-409547-2.12565-X.
63. Schissel, C. K. et al. Deep learning to design nuclear-targeting abiotic miniproteins. Nat. Chem. 13, 992-1000 (2021).
64. Bautista, A. D., Appelbaum, J. S., Craig, C. J., Michel, J. & Schepartz, A. Bridged β3-Peptide Inhibitors of p53-hDM2 Complexation: Correlation between Affinity and Cell Permeability. J. Am. Chem. Soc. 132, 2904-2906 (2010).
65. Harker, E. A. & Schepartz, A. Cell-Permeable β-Peptide Inhibitors of p53/hDM2 Complexation. ChemBioChem 10, 990-993 (2009).
66. Cheloha, R. W. et al. Consequences of Periodic α-to-β3 Residue Replacement for Immunological Recognition of Peptide Epitopes. ACS Chem. Biol. 10, 844-854 (2015).
67. Cheloha, R. W. et al. Recognition of Class II MHC Peptide Ligands That Contain β-Amino Acids. J. Immunol. 203, 1619-1628 (2019).
68. Melo Czekster, C., Robertson, W. E., Walker, A. S., Söll, D. & Schepartz, A. In Vivo Biosynthesis of a β-Amino Acid-Containing Protein. J. Am. Chem. Soc. 138, 5194-5197 (2016).
69. Ward, F. R., Watson, Z. L., Ad, O., Schepartz, A. & Cate, J. H. D. Defects in the Assembly of Ribosomes Selected for β-Amino Acid Incorporation. Biochemistry 58, 4494-4504 (2019).
70. Scott, T. A. et al. Widespread microbial utilization of ribosomal β-amino acid-containing peptides and proteins. Chem 8, 2659-2677 (2022).
71. Hamlish, N. X., Abramyan, A. M., Shah, B., Zhang, Z. & Schepartz, A. Incorporation of Multiple β2-Hydroxy Acids into a Protein In Vivo Using an Orthogonal Aminoacyl-tRNA Synthetase. ACS Cent. Sci. 10, 1044-1053 (2024).
72. Dunkelmann, D. L. et al. Adding α,α-disubstituted and β-linked monomers to the genetic code of an organism. Nature 625, 603-610 (2024).
73. Adaligil, E., Song, A., Hallenbeck, K. K., Cunningham, C. N. & Fairbrother, W. J. Ribosomal Synthesis of Macrocyclic Peptides with β2- and β2,3-Homo-Amino Acids for the Development of Natural Product-Like Combinatorial Libraries. ACS Chem. Biol. 16, 1011-1018 (2021).
74. Katoh, T. & Suga, H. Ribosomal Incorporation of Consecutive β-Amino Acids. J. Am. Chem. Soc. 140, 12159-12167 (2018).
75. Watson, Z. L. et al. Atomistic simulations of the Escherichia coli ribosome provide selection criteria for translationally active substrates. Nat. Chem. 15, 913-921 (2023).
76. Chin, J. W. Expanding and reprogramming the genetic code. Nature 550, 53-60 (2017).
77. Cruz-Navarrete, F. A. et al. β-Amino Acids Reduce Ternary Complex Stability and Alter the Translation Elongation Mechanism. ACS Cent. Sci. 10, 1262-1275 (2024).
78. Wan, W., Tharp, J. M. & Liu, W. R. Pyrrolysyl-tRNA synthetase: An ordinary enzyme but an outstanding genetic code expansion tool. Biochim. Biophys. Acta BBA—Proteins Proteomics1844, 1059-1070 (2014).
79. Huguenin-Dezot, N. et al. Trapping biosynthetic acyl-enzyme intermediates with encoded 2,3-diaminopropionic acid. Nature 565, 112-117 (2019).
80. Koch, N. G., Goettig, P., Rappsilber, J. & Budisa, N. Engineering Pyrrolysyl-tRNA Synthetase for the Incorporation of Non-Canonical Amino Acids with Smaller Side Chains. Int. J. Mol. Sci. 22, 11194 (2021).
81. Dawson, P. E. & Kent, S. B. H. Synthesis of Native Proteins by Chemical Ligation. Annu. Rev. Biochem. 69, 923-960 (2000).
82. Dose, C. & Seitz, O. New isocysteine building blocks and chemoselective peptide ligation. Org. Biomol. Chem. 2, 59-65 (2004).
83. Wang, C., Guo, Q.-X. & Fu, Y. Theoretical Analysis of the Detailed Mechanism of Native Chemical Ligation Reactions. Chem.-Asian J. 6, 1241-1251 (2011).
84. Tang, S. et al. Mechanism-based traps enable protease and hydrolase substrate discovery. Nature 602, 701-707 (2022).
85. Hall, M. P. et al. Engineered Luciferase Reporter from a Deep Sea Shrimp Utilizing a Novel Imidazopyrazinone Substrate. ACS Chem. Biol. 7, 1848-1857 (2012).
86. Volkwein, W., Maier, C., Krafczyk, R., Jung, K. & Lassak, J. A Versatile Toolbox for the Control of Protein Levels Using Na-Acetyl-1-lysine Dependent Amber Suppression. ACS Publicationshttps://pubs.acs.org/doi/pdf/10.1021/acssynbio.7b00048 (2017) doi:10.1021/acssynbio.7600048.
87. Leisle, L. et al. Cellular encoding of Cy dyes for single-molecule imaging. eLife 5, e19088 (2016).
88. Lee, J., Schwarz, K. J., Kim, D. S., Moore, J. S. & Jewett, M. C. Ribosome-mediated polymerization of long chain carbon and cyclic amino acids into peptides in vitro. Nat. Commun. 11, 4304 (2020).
89. Lajoie, M. J. et al. Genomically Recoded Organisms Expand Biological Functions. Science 342, 357-360 (2013).
90. Liu, C. C. & Schultz, P. G. Adding New Chemistries to the Genetic Code. Annu. Rev. Biochem. 79, 413-444 (2010).
91. Young, T. S. & Schultz, P. G. Beyond the Canonical 20 Amino Acids: Expanding the Genetic Lexicon. J. Biol. Chem. 285, 11039-11044 (2010).

### Supplementary Information

### 1-(6-nitrobenzo[d][1,3]dioxol-5-yl)ethan-1-one (12)

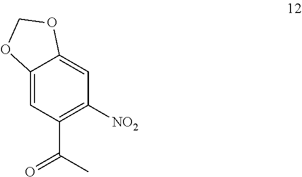

A solution of 3′,4′-(methylenedioxy) acetophenone (11) (3.283 g, 20 mmol) in glacial acetic acid (12.8 mL) was added dropwise to a 500 mL three-neck round-bottom flask containing conc. HNO₃ (27.2 mL, 70% w/w) at 0° C. over 1 h. The reaction mixture was maintained at 0° C. during the addition and for an additional 1 h with stirring under a N₂ atmosphere. The mixture was then warmed to 40° C. and stirred for an additional 3 h., the mixture was cooled to RT and poured into crushed ice in a beaker. A yellow precipitate appeared, which was stirred for 15 min and then filtered. The yellow solid was washed with water (3×50 mL) and dried under vacuum. The crude yellow solid was then purified by recrystallization (THF/n-hexane) to obtain the title compound (12) as yellow crystals.

### 1-(6-nitrobenzo[d][1,3]dioxol-5-yl)ethan-1-ol (±)-13)

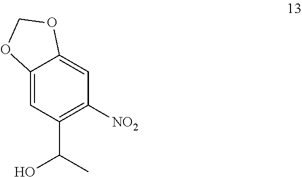

Ketone 12 (1.045 g, 5.0 mmol, 1.0 equiv) was dissolved in THF (25 mL) and NaBH₄ (472.87 mg, 12.5 mmol, 2.5 equiv) was added. The resultant suspension was stirred overnight at RT and then quenched with 1 N aq. HCl until gas evolution ceased. The mixture was poured into brine, the organic components were extracted with CH₂Cl₂ (3×), and the combined organic layers were dried over sodium sulfate and concentrated under reduced pressure. The residue was purified via flash column chromatography (100% CH₂Cl₂) to give the desired product as a yellow solid.

### 5-(1-bromoethyl)-6-nitrobenzo[d][1,3]dioxole ((±)-14)

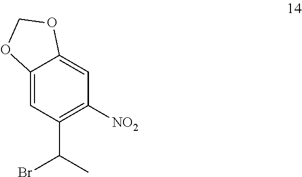

Alcohol (±)-13 (1.03 g, 4.88 mmol, 1.0 equiv) was dissolved in dry CH₂Cl₂ (24.5 mL) and cooled to 0° C. under a nitrogen atmosphere in a 250 mL round-bottom flask. After 10 min, PBr₃ (0.185 mL, 1.95 mmol, 0.4 equiv) was added dropwise at 0° C. Next, the mixture was brought to rt and stirred continuously for 16 h with aluminum foil wrapped around the flask. The reaction was judged to be complete by TLC analysis (SiO₂, TLC eluent: 100% CH₂Cl₂), cooled to 0° C., quenched by the addition of 1 M aq. NaOH (2 mL), and warmed to rt to stir for 30 min under a nitrogen atmosphere. After the quenching was complete, saturated aq. NaHCO₃ solution was added (20 mL). The contents were loaded into a separatory funnel, the aqueous phase was discarded, and the organic phase was washed sequentially with further saturated aq. NaHCO₃ solution (1× 20 mL) and brine (2× 20 mL). The organic layer was separated, dried over anhydrous Na₂SO₄, and concentrated in vacuo to obtain a yellow solid. The crude product was purified by flash chromatography on SiO₂ [eluent: 100% CH₂Cl₂] to obtain pure alkyl halide (±)-14 (1.33 g, 4.87 mmol, >99%) as yellow crystals.

### 2-((1-(6-nitrobenzo[d][1,3]dioxol-5-yl)ethyl)thio) ethan-1-ol ((±)-15)

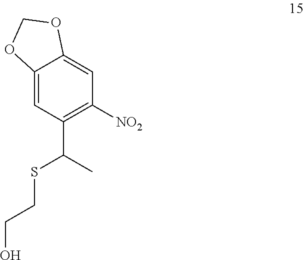 [SMILES: CC(SCCO)c1cc2c(cc1[N+](=O)[O-])OCO2]

An aqueous solution of NaOH was freshly prepared (0.5 M, 1.4 g in 7 mL of H₂O, 20.0 mmol, 5.8 equiv), loaded into a 100 mL round-bottom flask, and degassed by bubbling through a stream of nitrogen gas at rt. After 30 min, mercaptoethanol (0.254 mL, 3.6 mmol, 1.05 equiv) was added to the flask and degassing was continued for a further 15 min. Separately, freshly prepared alkyl halide (±)-14 (0.941 g, 3.43 mmol, 1.0 equiv) was dissolved in 1,4-dioxane (11.5 mL) in a 100 mL round-bottom flask wrapped in aluminum foil and degassed by bubbling through a stream of nitrogen gas for 15 min. The degassed solution of (±)-14 in 1,4-dioxane was added dropwise into the flask containing the aq. NaOH and mercaptoethanol solution, at rt under a positive pressure of nitrogen gas. The mixture was left stirring for 16 h at rt in the dark under a nitrogen atmosphere, after which time the reaction was judged to be complete by TLC. The mixture was then evaporated under reduced pressure to remove the volatile organic components. The resultant yellow aqueous mixture was then extracted with EtOAc (2×15 mL) and the combined organic phases were washed with a saturated NH₄Cl solution (1×15 mL), followed by brine (3×15 mL). The organic layer was then separated, dried over anhydrous Na₂SO₄, filtered, and evaporated to dryness to obtain a yellow oil. The product was purified by flash chromatography on SiO₂ (eluent: EtOAc/n-hexane=3:7) to obtain alcohol (±)-15 as yellow crystals.

### 2-((1-(6-nitrobenzo[d][1,3]dioxol-5-yl)ethyl)thio)ethyl 2,5-dioxopyrrolidine-1-carboxylate ((±)-16)

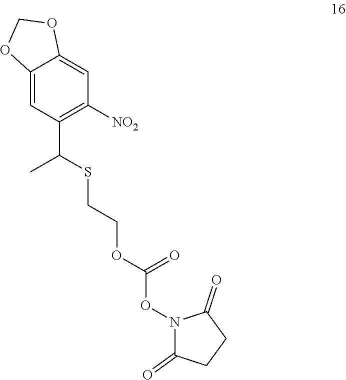 [SMILES: CC(SCCOC(=O)ON1C(=O)CCC1=O)c1cc2c(cc1[N+](=O)[O-])OCO2]

A 20 mL microwave vial was charged with alcohol (±)-15 (0.542 g, 1.99 mmol, 1.0 equiv) dissolved in dry CH₃CN (6 mL) under nitrogen atmosphere, and dry DIPEA (1.04 mL, 5.98 mmol, 3.0 equiv) was added. In a second 20 mL microwave vial N,N′-disuccinimidyl carbonate (0.716 g, 2.8 mmol, 1.4 equiv), was added with dry CH₃CN (6 mL) under nitrogen atmosphere (the mixture did not completely dissolve). The contents of the first vial were transferred to the second vial dropwise (12 mL total) under nitrogen atmosphere in the dark. After 30 min, all components were dissolved and left to stir at rt for 16 hours as a homogenous yellow solution. The reaction was judged to be complete by TLC analysis (SiO₂ plate, EtOAc/n-hexane=3:7) after this time. Carbonate (±)-16 was immediately carried to the next step without further purification: R_{f}=0.12 (SiO₂ plate, EtOAc/n-hexane=3:7).

### L-isoserine methyl ester (H-isoS-OMe, 17)

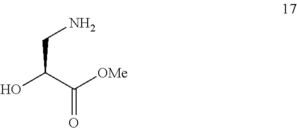

To a solution of isoserine ((S)-3-amino-2-hydroxypropanoic acid, 139 mg, 1.3 mmol, 1.0 equiv) in anhydrous methanol (3 mL) was added TMSCl (0.33 mL, 2.6 mmol, 2 equiv). After 24 h, the mixture was concentrated in vacuo to provide methyl ester 17 as a colorless solid.

### N-Photo-Masked-L-Isoserine Methyl Ester (Pm-isoS-OMe, 18)

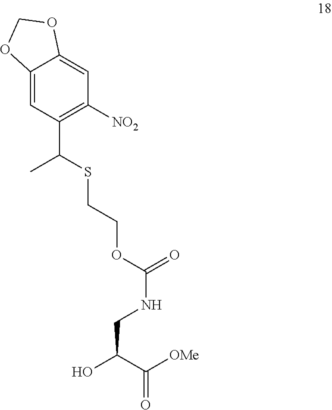 [SMILES: COC(=O)[C@@H](O)CNC(=O)OCCSC(C)c1cc2c(cc1[N+](=O)[O-])OCO2]

Methyl ester 17 (51 mg, 0.5 mmol, 1.1 equiv) was added in one portion to a solution of carbonate (±)-16 (0.44 mmol, 1.0 equiv, prepared as described above) in dry CH₃CN (2.2 mL) under nitrogen gas and stirred for 16 h at rt. After this time, the reaction was judged to be complete by LC-MS and the reaction mixture was diluted with water containing 0.1% TFA, filtered, and purified by RP-HPLC while shielded from light. The residue was then subjected to preparative reverse phase HPLC on a Waters Prep 150 LC System [CSH C18 19×150 mm OBD Column 5 μm; gradient: 5-50% H₂O—CH₃CN (±0.1% TFA throughout) mobile phase over 16 min] to obtain the title compound. Purity of resulting fractions was determined by LC-MS and pure fractions were immediately evaporated of solvent, affording photo-masked compound 18 as a yellow oil and a mixture of diastereomers (62 mg, 34% yield). The product was immediately used in the subsequent reaction to avoid possible decay.

### N-Photo-Masked-O—(N-Acetylglycyl)-L-Isoserine Methyl Ester (Pm-isoS (Ac-Gly)-OMe, 4)

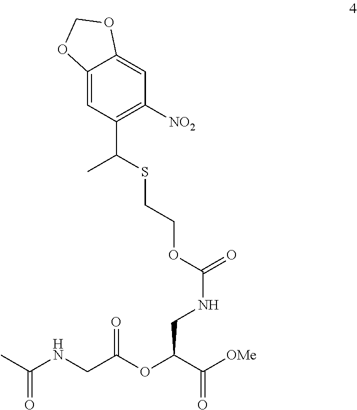 [SMILES: COC(=O)[C@H](CNC(=O)OCCSC(C)c1cc2c(cc1[N+](=O)[O-])OCO2)OC(=O)CNC(C)=O]

To a solution of N-acetylglycine (68 mg, 0.6 mmol, 10 equiv) in dry CH₂Cl₂ (5 mL) was added DIC (0.09 mL, 0.6 mmol, 10 equiv) and NMI (0.05 mL, 0.6 mmol, 10 equiv). The resulting mixture was added to a flask containing photo-masked compound 18 (24 mg, 0.06 mmol, 1.0 equiv). After stirring for 0.5 h, the reaction mixture was concentrated in vacuo. The crude material was dissolved (95% water, 5% MeCN, 0.1% TFA), filtered through a 0.2 micron PTFE filter, and purified by reverse phase HPLC on a Waters Prep 150 LC System [CSH C18 19×150 mm OBD Column 5 μm; gradient: 5-50% H₂O—CH₃CN (±0.1% TFA throughout) mobile phase over 30 min] to afford a waxy yellow solid as a mixture of diastereomers (16 mg, 53% yield).

### 2-(2-oxo-1-oxa-4-thiaspiro[4.4]nonan-3-yl) acetic acid ((±)-20)

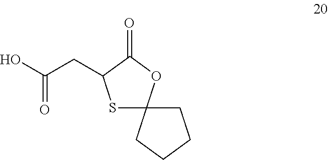 [SMILES: O=C(O)CC1SC2(CCCC2)OC1=O]

To a two-neck round-bottom flask equipped with a stir bar was added 2-mercaptosuccinic acid (±)-19 (7.00 g, 46.6 mmol, 1.0 equiv) and p-toluenesulfonic acid monohydrate (0.89 g, 4.66 mmol, 0.1 equiv). The flask was charged with benzene (100 mL), and a Dean-Stark apparatus was attached and charged with additional benzene and a small aliquot of water. The Dean-Stark apparatus was affixed with a reflux condenser, and the system was sealed with rubber septa and placed under a nitrogen atmosphere. The suspension was briefly stirred (<2 min), and distilled cyclopentanone (6.2 mL, 69.9 mmol, 1.5 equiv) was added to the flask through a septum via syringe. The reaction was placed into a preheated oil bath and refluxed for 4.5 h, at which point the solution had turned clear. The reaction was cooled to room temperature and concentrated in vacuo. The crude residue was dissolved in saturated aq. NaHCO₃ and washed with DCM (3×). The combined organic extracts were discarded and the aqueous phase was acidified to pH 1-2 with concentrated aq. HCl and extracted with DCM (3×). The combined organic phases were then dried over Na₂SO₄, filtered, and concentrated in vacuo. The dried material was then purified by recrystallization in EtOAc/Hexanes to afford thioketal (±)-20 as an off-white solid (3.17 g, 14.7 mmol, 31% yield).

### N-Photo-Masked DL-Isocysteine Thioketal ((±)-21)

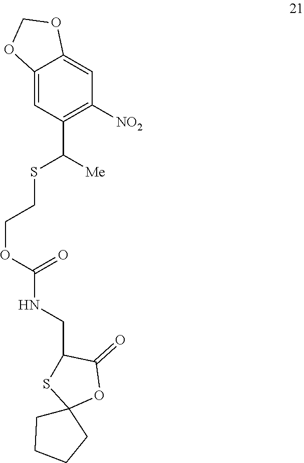 [SMILES: CC(SCCOC(=O)NCC1SC2(CCCC2)OC1=O)c1cc2c(cc1[N+](=O)[O-])OCO2]

To a round-bottom flask equipped with a stir bar was added thioketal (±)-20 (3.17 g, 14.7 mmol, 1.0 equiv) and dry toluene (59 mL). The flask was sealed with a rubber septum and placed under a nitrogen atmosphere. Triethylamine (2.45 mL, 17.6 mmol, 1.2 equiv) was added slowly at room temperature through the septum via syringe, and the solution was stirred for 30 min. The solution was then cooled to 0° C. with an ice bath, and diphenylphosphoryl azide (3.79 mL, 17.6 mmol, 1.2 equiv) was added slowly through the septum via syringe. The solution was removed from the ice bath and stirred for 3.5 h at room temperature, during which time it gradually darkened to a caramel color. The reaction mixture was then opened to air via a vent needle in the septum, warmed to 85° C. in a preheated oil bath, and stirred with a vent needle until gas evolution ceased (ca. 30 min). The flask was cooled to 60° C., at which point alcohol (±)-15 (3.78 g, 13.9 mmol, 0.95 equiv) was added slowly via syringe. The solution was then stirred overnight at 60° C., cooled to room temperature, and diluted with EtOAc. The mixture was then washed sequentially with saturated aq. NaHCO₃ (1×), water (1×), 10% aq. citric acid (1×), and brine (1×). The organic phase was dried over Na₂SO₄, filtered, and concentrated in vacuo. The crude residue was purified twice via normal phase automated flash column chromatography (0-100% EtOAc/Hexanes to 0-10% MeOH/EtOAc), twice by reverse phase automated flash column chromatography (0-100% MeCN/H₂O), and lyophilized to afford photo-masked thioketal (±)-21 as a viscous yellow oil and as a mixture of diastereomers (2.60 g, 5.37 mmol, 36% yield).

### N-photo-masked S—(N-acetylglycyl) DL-isocysteine methyl ester (pm-^{DL}isoC(Ac-Gly)-OMe, (±)-5)

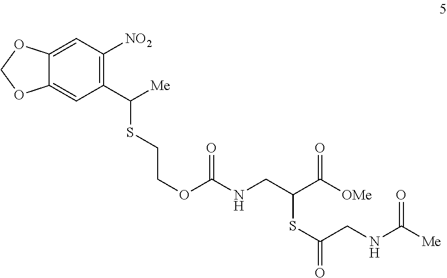 [SMILES: COC(=O)C(CNC(=O)OCCSC(C)c1cc2c(cc1[N+](=O)[O-])OCO2)SC(=O)CNC(C)=O]

To a round-bottom flask equipped with a stir bar was added was added photo-masked thioketal (±)-21 (576 mg, 1.19 mmol, 1 equiv) and MeOH (8 mL). Solid NaOH (99 mg, 2.48 mmol, 2.1 equiv) was then added to the flask in one portion. The reaction was vigorously stirred for 1 h, after which the solution was diluted with water and acidified to pH ~1 with concentrated aq. HCl. The solution was then extracted with DCM (3×), and the combined organic layers were washed with brine, dried over Na₂SO₄, filtered, and concentrated in vacuo to provide the wet intermediate thiol (±)-8 as a viscous oil (503 mg). A portion of the crude material (460 mg, 1.06 mmol) was used in the next step without further purification. A crude 1H NMR spectrum of this intermediate compound is provided for reference.

To a vial equipped with a stir bar was added N-acetylglycine (150 mg, 1.28 mmol, 1.2 equiv) and HATU (485 mg 1.28 mmol, 1.2 equiv). The vial was then charged with DMF (3.5 mL), and dry DIPEA (0.56 mL, 3.19 mmol, 3 equiv) was added dropwise with stirring. The solution was pre-stirred for 10 min, then transferred to a vial containing thiol (±)-8 (460 mg, 1.06 mmol, 1 equiv) and a stir bar. The resultant solution was then stirred for 2.5 h and partitioned between EtOAc (ca. 60 mL) and 5% aq. LiCl (ca. 60 mL). The organic phase was then washed sequentially with 5% aq. LiCl (3×), 10% aq. citric acid (1×), saturated aq. NaHCO₃ (2×), and brine (1×). The organic phase was dried over Na₂SO₄, filtered, and concentrated in vacuo. The resultant material was then purified via normal phase automated flash column chromatography (20-100% EtOAc/Hexanes) and lyophilized to afford photo-masked thioester (±)-5 as a yellow foam and as a mixture of diastereomers (324 mg, 0.610 mmol, 51% yield over two steps, uncorrected for diverted material).

### Ac-Gly-isoS-OH (22)

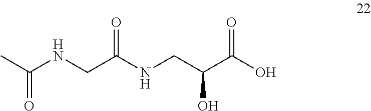

The isoserine authentic standard was prepared using solid-phase peptide synthesis (SPPS). 2-chlorotrityl chloride resin (143 mg, 1.4 mmol/g, 1.0 equiv) was transferred to a 5 mL fritted syringe and swelled with DCM for five minutes. Solvent was evacuated and replaced with a solution of (2S)-3-((((9H-fluoren-9-yl) methoxy) carbonyl)amino)-2-(tert-butoxy) propanoic acid (0.3 mmol, 1.5 equiv) and DIPEA (0.8 mmol, 4 equiv) in DCM and incubated for 1 hour. This solution was discarded, and the resin was washed three times with DMF. The resin was then capped with a solution of DCM: MeOH: DIPEA (17:2:1, v/v) for 30 minutes. The resin was washed three times with DMF, deprotected using 20% piperidine in DMF for 10 minutes, and washed again three times with DMF. A solution of N-acetylglycine (2 mmol, 10 equiv), HBTU (1.9 mmol, 9.5 equiv), and DIPEA (4 mmol, 20 equiv) in DMF was added to the syringe and incubated for 30 minutes. The resin was then washed three times with DMF and three times with DCM before being fully dried. Finally, the resin was cleaved using a solution of TFA: water (98:2) for two hours. The isolated solution was dried in vacuo, to yield dipeptide 22 as a colorless oil (37 mg, 93% from resin loading).

### Ac-Gly-isoS-OMe (6)

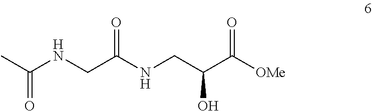

To a solution of dipeptide 22 (34 mg, 0.17 mmol, 1.0 equiv) in dry methanol (3 mL) was added TMSCl (0.04 mL, 0.33 mmol, 2.0 equiv). After stirring for 16 h, the reaction mixture was concentrated in vacuo to afford the desired dipeptide methyl ester 6 as a colorless oil (36 mg, quant.) 1H NMR (600 MHz, methanol-d₄) δ=4.56 (s, 1H), 4.26 (dd, J=6.4, 4.8 Hz, 1H), 3.83 (d, J=1.4 Hz, 2H), 3.56 (dd, J=13.7, 4.8 Hz, 1H), 3.45 (dd, J=13.7, 6.4 Hz, 1H), 3.35 (s, 3H), 2.00 (s, 3H). ¹³C{¹H} NMR (151 MHz, methanol-d₄) δ=174.4, 173.9, 172.1, 70.7, 43.8, 43.5, 30.7, 22.4. MS (ESI) m/z calcd, for C₈H₁₅N₂O₅⁺ [MH]+: 219.10. found: 219.10.

### S-Trt-DL-isocysteine (H-DLisoC (Trt)-OH, (±)-24)

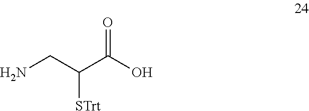

Thioketal (±)-20, an intermediate en route to compound (±)-24, was prepared as described previously (vide supra) with the following modifications: (i) the compound was prepared at a 59.9 mmol scale; (ii) the reaction time was shortened to 3.5 h; (iii) MgSO₄ was used as the drying agent, rather than Na₂SO₄; and (iv) the material was not subjected to purification via recrystallization. The resultant off-white solid (thioketal (±)-20, 1.99 g, 9.20 mmol) was used in the next step without further purification. A crude ¹H NMR spectrum is provided for reference; the product peaks are in reasonable accord with the literature characterization3.

To a round-bottom flask equipped with a stir bar was added crude thioketal (±)-20 (1.99 g, 9.20 mmol, 1.0 equiv) and dry toluene (40 mL). The flask was sealed with a rubber septum and placed under a nitrogen atmosphere. Triethylamine (1.5 mL, 10.8 mmol, 1.2 equiv) was added slowly through the septum via syringe at room temperature, and the solution was stirred for 30 min. The solution was then cooled to 0° C. with an ice bath, and diphenylphosphoryl azide (2.18 mL, 10.1 mmol, 1.1 equiv) was added slowly through the septum via syringe. The resultant solution was removed from the ice bath and stirred for 4 h at room temperature, during which it gradually darkened to a caramel color. The reaction mixture was then opened to air via a vent needle in the septum, warmed to 85° C. in a preheated oil bath, and stirred with a vent needle until gas evolution ceased (ca. 30 min). The flask was then removed from the oil bath and cooled to room temperature. The reaction mixture was diluted with EtOAc (40 mL), washed with saturated aq. NaHCO₃ (1×), and then washed with DI water (2×). The organic phase was dried over MgSO₄, filtered, and concentrated to provide a crude residue, which was used in the next step without further purification.

The crude residue from the previous step was taken up in 6 N aq. HCl (40 mL) and refluxed for 3 h. The reaction mixture was then cooled to room temperature and washed with Et₂O (3×). The ethereal washes were discarded, and the aqueous phase was concentrated in vacuo to provide a dark red oil ((±)-23), which was used in the next step without further purification.

Trifluoroacetic acid (33 mL) was added to the crude dark red oil ((±)-23) from the previous step, and the solution was briefly stirred before triphenylmethanol (2.58 g, 9.9 mmol, 1.1 equiv) was added portionwise. The resultant mixture was stirred for 1 h at room temperature and concentrated in vacuo. The residue was then dissolved in Et₂O (33 mL), and 0.2 N aq. NaOAc was added with vigorous stirring until the biphasic mixture had a pH of ~4 and a tan precipitate appeared (ca. 500 mL of 0.2 N aq. NaOAc was required). The mixture was filtered, and the precipitate was dried under vacuum. The precipitate was then collected and dissolved in acetone (33 mL) and stirred for 30 min at 40° C. The mixture was cooled to room temperature and placed in a −20° C. freezer overnight. The mixture was filtered and the precipitate was dried under vacuum. An additional crop could be obtained by analogous recrystallization of the initial filtrate from NaOAc treatment. In total, 1.397 g (3.84 mmol) of crude trityl-protected isocysteine ((±)-24) was obtained from this process as an off-white solid, and a portion of the material (0.477 g, 1.31 mmol) was carried forward to the next step without further purification. A crude 1H NMR spectrum is provided for reference; the product peaks are in reasonable accord with the literature characterization4.

### N-Fmoc-S-Trt-DL-isocysteine (Fmoc-^{DL}isoC(Trt)-OH, (±)-25)

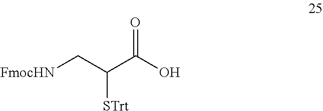

A round-bottom flask equipped with a stir bar was charged with crude trityl-protected isocysteine (±)-24 (477 mg, 1.31 mmol, 1.00 equiv), 1,4-dioxane (3.3 mL), and water (1.7 mL) to afford a suspension. NaHCO₃ (116 mg, 1.38 mmol, 1.05 equiv) was added, and the mixture was stirred for 20 min. Fmoc-OSu (450 mg, 1.33 mmol, 1.02 equiv) was then added, and the mixture was stirred for an additional 30 min, at which point an additional portion of 1,4-dioxane (3.3 mL) and water (1.7 mL) was added. The reaction was stirred overnight at room temperature. The resultant mixture was diluted with water (10 mL), affording a cloudy white suspension. The mixture was concentrated in vacuo to approximately ½ of the original volume, and decanted. The decanted solid was then split into three sets and purified via reverse phase automated flash column chromatography (Set 1:0-100% MeCN in H₂O; Set 2:10-100% MeCN in H₂O; Set 3:5-100% MeCN in H₂O+5% of aq. 2% formic acid buffer throughout. Vide infra). The residue was taken up in 10% aq. citric acid (50 mL) and vigorously stirred for 2.5 hours and extracted with EtOAc (3×30 mL). The combined organic phases were washed with brine (1×), dried over Na₂SO₄, filtered, and concentrated in vacuo. The resultant material was filtered through a syringe filter (0.2 μm pore size) with DCM/MeCN/Et₂O (1:1:1, ca. 10 mL total volume), concentrated in vacuo, purified via reverse phase automated flash column chromatography (2-100% MeCN in H₂O+5% of aq. 2% formic acid buffer throughout), and lyophilized to afford Fmoc-DLisoC (Trt)-OH (compound (±)-25, 227 mg, 0.388 mmol, 0.6% yield over five steps, uncorrected for diverted material).

### Ac-Gly-^{DL}isoC-OH ((±)-26)

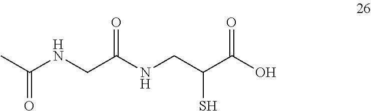

The isocysteine (isoC) authentic standard was prepared using solid-phase peptide synthesis (SPPS). 2-chlorotrityl chloride resin (36 mg, 1.4 mmol/g, 1 equiv) was transferred to a 5 mL fritted syringe and swelled with DCM for five minutes. Solvent was evacuated and replaced with a solution of Fmoc-isoC (Trt)-OH (compound (±)-25, 59 mg, 0.1 mmol, 2 equiv) and DIPEA (0.03 mL, 0.2 mmol, 4 equiv) in DCM and incubated for 1 hour. This solution was discarded, and the resin was washed three times with DMF. The resin was then capped with a solution of DCM: MeOH: DIPEA (17:2:1, v/v) for 30 minutes. The resin was washed three times with DMF, deprotected using 20% piperidine in DMF for 10 minutes, and washed again three times with DMF. A solution of N-acetylglycine (59 mg, 0.5 mmol, 10 equiv), HBTU (180 mg, 0.48 mmol, 9.5 equiv), and DIPEA (0.17 mL, 1 mmol, 20 equiv) in DMF was added to the syringe and incubated for 30 minutes. The resin was then washed three times with DMF and three times with DCM before being fully dried. Finally, the resin was cleaved using a solution of TFA: water: TIPS (95:3:2) for two hours. The isolated solution was dried in vacuo, and the residue was diluted with 5% acetonitrile in water, filtered through a 0.2 micron PTFE filter, and purified by reverse phase HPLC on a Waters Prep 150 LC System [CSH C18 19×150 mm OBD Column 5 μm; gradient: 5-50% H₂O—CH₃CN (±0.1% TFA throughout) mobile phase over 30 min] to yield dipeptide (±)-26 as a colorless solid (8 mg, 73% from resin loading), which was passed forward to the next step.

### Ac-Gly-^{DL}isoC-OMe ((±)-7)

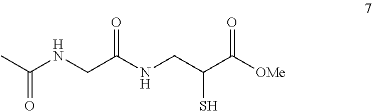

To a solution of dipeptide (±)-26 (6 mg, 0.03 mmol, 1.0 equiv) in dry methanol (5 mL) was added TMSCl (0.01 mL, 0.05 mmol, 2.0 equiv). After stirring for 48 h, the reaction mixture was concentrated in vacuo to afford the desired dipeptide methyl ester (±)-7 as a colorless solid (5.7 mg, 0.03 mmol, >99%).

### N_{α}-Boc-N_{β}-Photo-Masked-L-Diaminopropionic Acid (27)

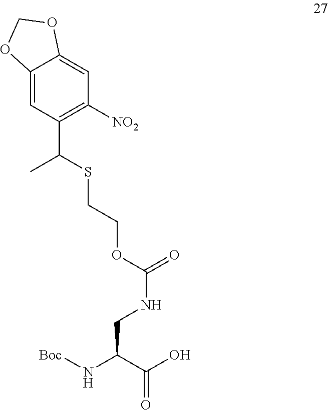 [SMILES: CC(SCCOC(=O)NC[C@H](NC(=O)OC(C)(C)C)C(=O)O)c1cc2c(cc1[N+](=O)[O-])OCO2]

Boc-L-Dap-OH (0.441 g, 2.16 mmol, 1.08 equiv) was added in one portion to a solution of 16 (1.99 mmol, 1.00 equiv, prepared as described above) in dry CH₃CN (12 mL) under nitrogen gas and stirred for 16 h at rt. After this time, the reaction was judged to be complete by LC-MS and the reaction mixture was concentrated in vacuo. The residue was then subjected to preparative reverse phase HPLC on a Waters Prep 150 LC System [CSH C18 19×150 mm OBD Column 5 μm; gradient: 20-50% H₂O—CH₃CN (±0.1% TFA throughout) mobile phase over 16 min] to obtain photo-masked Boc-L-Dap-OH (27) as a yellow solid (0.947 g, 95%) and a mixture of ~1:1 epimers.

### N_{β}-Photo-Masked-L-Diaminopropionic Acid (pm-DAP, 1)

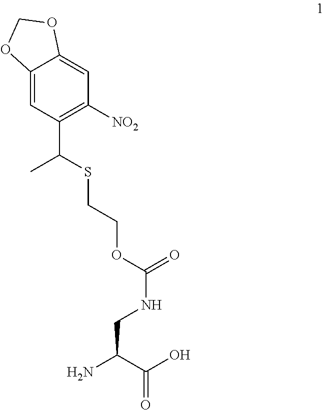 [SMILES: CC(SCCOC(=O)NC[C@H](N)C(=O)O)c1cc2c(cc1[N+](=O)[O-])OCO2]

Photo-masked Boc-L-Dap-OH (27) (0.412 g, 0.822 mmol, 1.0 equiv) was added to a dry 20 mL microwave vial and dissolved in 70% v/v TFA in CH₂Cl₂ (5 mL). The flask was wrapped in foil to exclude light. The yellow reaction mixture was left stirring at rt in the dark. After 2 h, the reaction was judged to be complete by LC-MS analysis. The reaction mixture was concentrated under reduced pressure to obtain a yellow gum. This residue was then subjected to preparative reverse phase HPLC on a Waters Prep 150 LC System [CSH C18 19×150 mm OBD Column 5 μm; gradient: 15-65% H₂O—CH₃CN (±0.1% TFA throughout) mobile phase over 16 min] to obtain side-chain photo-masked diaminopropionic acid (pm-DAP, 1) as a yellow solid (0.947 g, 72%) and a mixture of ~1:1 epimers.

### N-Photo-Masked-L-Isoserine (pm-isoS, 2)

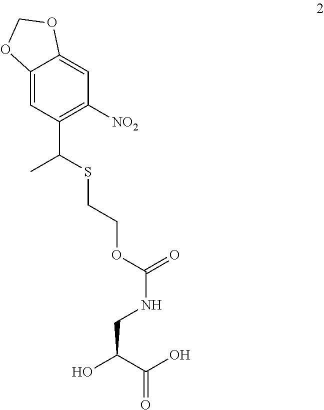 [SMILES: CC(SCCOC(=O)NC[C@H](O)C(=O)O)c1cc2c(cc1[N+](=O)[O-])OCO2]

L-isoserine (0.229 g, 2.16 mmol, 1.08 equiv) was added in one portion to a solution of 16 (1.99 mmol, 1.00 equiv, prepared as described above) in dry CH₃CN (12 mL) under nitrogen gas and stirred for 16 h at 60° C. After this time the reaction was judged to be complete by LC-MS and the contents dried under reduced pressure. This was then subjected to preparative reverse phase high-performance liquid chromatography (RP-HPLC) on a Waters Prep 150 LC System [CSH C₁₈ 19×150 mm OBD Column 5 μm; gradient: 20-50% H₂O—CH₃CN (±0.1% TFA throughout) mobile phase over 16 min] to obtain the desired photo-masked isoserine (pm-isoS, 2) as a yellow solid (0.793 g, >99%) and a mixture of ~1:1 epimers.

### N-Photo-Masked-DL-Isocysteine (pm-isoC, (±)-3)

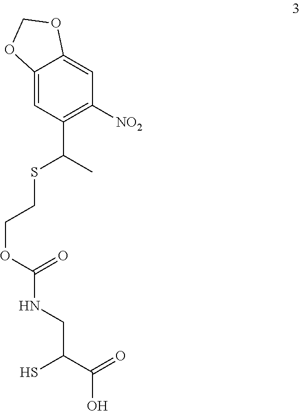 [SMILES: CC(SCCOC(=O)NCC(S)C(=O)O)c1cc2c(cc1[N+](=O)[O-])OCO2]

Thioketal (±)-20 (530 mg, 1.1 mmol, 1 equiv) was dissolved in 2 mL THF and 1 M LiOH (2 mL) was added dropwise at 0° C. The mixture was warmed to room temperature and stirred vigorously for 3 h with aluminum foil wrapped around the vial. The reaction was judged to be complete by LC-MS analysis. The reaction was then acidified to pH 5 with 1 N aq. HCl, extracted with EtOAc (3×5 mL), and the organic phase was dried over sodium sulfate and concentrated under reduced pressure. The residue was then subjected to preparative reverse phase HPLC on a Waters Prep 150 LC System [CSH C18 19×150 mm OBD Column 5 μm; gradient: 5-95% H₂O—CH₃CN (+0.1% TFA throughout) mobile phase over 16 min] to obtain the desired photo-masked isocysteine (pm-isoC, (±)-3) as a yellow gum (0.456 g, >99%) and a mixture of ~1:1 epimers.

1. A method of protein backbone editing, the method comprising:
a) providing a cell engineered to express an orthogonal aminoacyl-tRNA synthetase under conditions wherein the synthetase accepts an α-hydroxy acid or α-thiol acid monomer comprising a side chain bearing a masked nucleophile, and the monomer is incorporated into a protein translated in the cell,
b) post-translationally unmasking the nucleophile of the incorporated monomer, wherein the unmasked monomer engages in a thermodynamically favored intramolecular Backbone Extension Acyl Rearrangement (BEAR) reaction that edits the protein backbone.

2. The method of claim 1, wherein the protein comprises a non-α-backbone comprising a non-α-peptide linkage, including an additional R group on a sidechain or an alternative hetero-atom, selected from S, P, O and N, in the linkage.

3. The method of claim 1, wherein the BEAR reaction is used to generate a protein containing one or two copies of a β-backbone, a γ-backbone, or a δ-backbone.

4. The method of claim 1, wherein the protein comprises a β2-backbone comprising a β2-peptide linkage.

5. The method of claim 1, wherein the protein comprises a γ-backbone comprising a γ-peptide linkage.

6. The method of claim 1, wherein the protein comprises a δ-backbone comprising a δ-peptide linkage.

7. The method of claim 1, wherein the protein comprises a plurality of peptides linkages selected from β2-, γ- and δ-peptide linkages.

8. The method of claim 1, wherein the aminoacyl-tRNA synthetase is an orthogonal synthetase which incorporates the BEAR monomer that is an alpha-hydroxy acid with a nucleophile on the side chain in the BEAR reaction.

9. The method of claim 1, wherein the aminoacyl-tRNA synthetase is a pyrrolysyl-tRNA synthetase (PylRS).

10. The method of claim 1, wherein the aminoacyl-tRNA synthetase is a pyrrolysyl-tRNA synthetase (PylRS) of an archaea or methanogenic bacteria, wild-type or engineered.

11. The method of claim 1, wherein the aminoacyl-tRNA synthetase is a pyrrolysyl-tRNA synthetase (PylRS) of Methanosarcina mazei (Mm), Methanosarcina barkeri (Mb) or Methanocarcina Alvus (Ma), wild-type or engineered.

12. The method of claim 1, wherein the aminoacyl-tRNA synthetase is selected from: highly polyspecific pyrrolysyl-tRNA synthetase” (HpRS), chimeric orthogonal aminoacyl-tRNA synthetase/tRNA pair, Mb PylRS variant DAPRS and engineered aminoacyl-tRNA synthetases.

13. The method of claim 1, wherein the nucleophile is a carbon or nitrogen atom, including secondary and tertiary amine and carbon nucleophiles.

14. The method of claim 1, wherein the unmasking is effected with light, heat, pH, reduction, oxidation, or click chemistry, in a biologically compatible chemical reaction.

15. The method of claim 1, wherein the masking is effected with photomasking or chemical reductive masking.

16. The method of claim 1, wherein the cell is a bacterial, yeast or mammalian cell.

17. The method of claim 1, wherein the cell is E. coli, S. cerevisiae or human.

18. A method of protein backbone editing, the method comprising:
using a proximity-guided intramolecular rearrangement that edits the protein backbone post-translationally, wherein an orthogonal aminoacyl-tRNA synthetase enzyme accepts diverse monomers, including those whose side chains contain masked nucleophiles,
wherein a monomer whose side chains contains a masked nucleophile is introduced into the protein translated in vivo, followed by nucleophile unmasking, to provide a thermodynamically favored intramolecular Backbone Extension Acyl Rearrangement (BEAR) reaction that edits the protein backbone to install an extended backbone monomer.

19. The method of claim 18, wherein the protein comprises a non-α-backbone comprising a non-α-peptide linkage, including an additional R group on a sidechain or an alternative hetero-atom, selected from S, P, O and N, in the linkage.

20. The method of claim 18, wherein the BEAR reaction is used to generate a protein containing one or two copies of a β-backbone, a γ-backbone, or a δ-backbone.
